(19) United States
(12) Patent Application Publication
(10) Pub. No.: US 2006/0089405 A1
(43) Pub. Date: Apr. 27, 2006

(54) Asymmetric synthesis of dihydrobenzofuran derivatives

(75) Inventors: Dahui Zhou, East Brunswick, NJ (US); Gary Paul Stack, Ambler, PA (US); Alexander V. Gontcharov, Rivervale, NJ (US)

Correspondence Address: CHOATE, HALL & STEWART LLP, PATENT GROUP, TWO INTERNATIONAL PLACE, BOSTON, MA, 02110, US

(73) Assignee: Wyeth, Madison, NJ (US)

(21) Appl. No.: 11/255,562

(22) Filed: Oct. 21, 2005

Related U.S. Application Data
(60) Provisional application No. 60/621,023, filed on Oct. 21, 2004.

(51) Int. Cl. C07D 307/87; A61K 31/343; C07F 1/08

(52) U.S. Cl. 514/469; 549/467

(57) ABSTRACT

This invention concerns a process for the preparation of benzofuran derivatives. In some aspects, these compounds are of formula I: 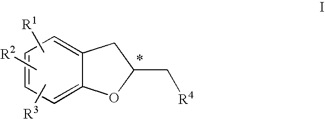
wherein each of R¹, R², R³, and R⁴ is as defined herein.

### CROSS-REFERENCE TO RELATED APPLICATIONS

This application claims priority to U.S. provisional application No. 60/621,023, filed Oct. 21, 2004, the entire contents of which are hereby incorporated herein by reference.

### FIELD OF THE INVENTION

The present invention concerns processes for the asymmetric synthesis of dihydrobenzofuran derivatives.

### BACKGROUND OF THE INVENTION

Schizophrenia affects approximately 5 million people. The most prevalent treatments for schizophrenia are currently the ‘atypical’ antipsychotics, which combine dopamine (D₂) and serotonin (5-HT_{2A}) receptor antagonism. Despite the reported improvements in efficacy and side-effect liability of atypical antipsychotics relative to typical antipsychotics, these compounds do not appear to adequately treat all the symptoms of schizophrenia and are accompanied by problematic side effects, such as weight gain (Allison, D. B., et. al., Am. J. Psychiatry, 156: 1686-1696, 1999; Masand, P. S., Exp. Opin. Pharmacother. 1: 377-389, 2000; Whitaker, R., Spectrum Life Sciences. Decision Resources. 2:1-9, 2000).

Atypical antipsychotics also bind with high affinity to 5-HT_{2C} receptors and function as 5-HT_{2C} receptor antagonists or inverse agonists. Weight gain is a problematic side effect associated with atypical antipsychotics such as clozapine and olanzapine, and it has been suggested that 5-HT_{2C} antagonism is responsible for the increased weight gain. Conversely, stimulation of the 5-HT_{2C} receptor is known to result in decreased food intake and body weight (Walsh et. al., Psychopharmacology 124: 57-73, 1996; Cowen, P. J., et. al., Human Psychopharmacology 10: 385-391, 1995; Rosenzweig-Lipson, S., et. al., ASPET abstract, 2000).

Several lines of evidence support a role for 5-HT_{2C} receptor agonism or partial agonism as a treatment for schizophrenia. Studies suggest that 5-HT_{2C} antagonists increase synaptic levels of dopamine and may be effective in animal models of Parkinson's disease (Di Matteo, V., et. al., Neuropharmacology 37: 265-272, 1998; Fox, S. H., et. al., Experimental Neurology 151: 35-49, 1998). Since the positive symptoms of schizophrenia are associated with increased levels of dopamine, compounds with actions opposite to those of 5-HT_{2C} antagonists, such as 5-HT_{2C} agonists and partial agonists, should reduce levels of synaptic dopamine. Recent studies have demonstrated that 5-HT_{2C} agonists decrease levels of dopamine in the prefrontal cortex and nucleus accumbens (Millan, M. J., et. al., Neuropharmacology 37: 953-955, 1998; Di Matteo, V., et. al., Neuropharmacology 38: 1195-1205, 1999; Di Giovanni, G., et. al., Synapse 35: 53-61, 2000), brain regions that are thought to mediate critical antipsychotic effects of drugs like clozapine. However, 5-HT_{2C} agonists do not decrease dopamine levels in the striatum, the brain region most closely associated with extrapyramidal side effects. In addition, a recent study demonstrates that 5-HT_{2C} agonists decrease firing in the ventral tegmental area (VTA), but not in the substantia nigra. The differential effects of 5-HT_{2C} agonists in the mesolimbic pathway relative to the nigrostriatal pathway suggest that 5-HT_{2C} agonists have limbic selectivity, and will be less likely to produce extrapyramidal side effects associated with typical antipsychotics.

Certain dihydrobenzofurans are believed to possess affinity for the 5HT_{2C} receptor. Preferably, such dihydrobenzofurans act as agonists or partial agonists at the 5HT_{2C} receptor and therefore are believed to be useful in a variety of medicinal applications, for example, as discussed above. The present invention provides stereoselective methods for synthesizing dihydrobenzofurans.

### SUMMARY OF THE INVENTION

As described herein, the present invention provides methods for preparing compounds having activity as 5HT_{2C} agonists or partial agonists. These compounds are useful for treating disorders including schizophrenia, schizophreniform disorder, schizoaffective disorder, delusional disorder, substance-induced psychotic disorder, L-DOPA-induced psychosis, psychosis associated with Alzheimer's dementia, psychosis associated with Parkinson's disease, psychosis associated with Lewy body disease, dementia, memory deficit, intellectual deficit associated with Alzheimer's disease, bipolar disorders, depressive disorders, mood episodes, anxiety disorders, adjustment disorders, eating disorders, epilepsy, sleep disorders, migraines, sexual dysfunction, gastrointestinal disorders, obesity, or a central nervous system deficiency associated with trauma, stroke, or spinal cord injury. Such compounds include those of formula II: 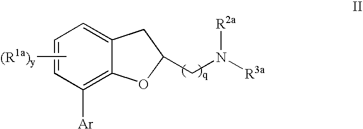
or a pharmaceutically acceptable salt thereof, wherein each of R^{1a}, R^{2a}, R^{3a}, Ar, q, and y is as defined herein.

The present invention also provides synthetic intermediates useful for preparing such compounds.

### DETAILED DESCRIPTION OF CERTAIN EMBODIMENTS

The methods and intermediates of the present invention are useful for preparing compounds as described in, e.g. U.S. patent application entitled “Dihydrobenzofuranyl Alkanamine Derivatives and Methods for Using Same,” filed in the name of Jonathan Gross, et al., having U.S. Ser. No. 11/113,170, filed Apr. 22, 2005, and claiming benefit to U.S. application Ser. No. 10/970,014, filed Oct. 21, 2004, and U.S. provisional application 60/514,454, filed on Oct. 24, 2003, each of which is hereby incorporated herein by reference in its entirety for all purposes. In certain embodiments, the present compounds are generally prepared according to Scheme I set forth below:

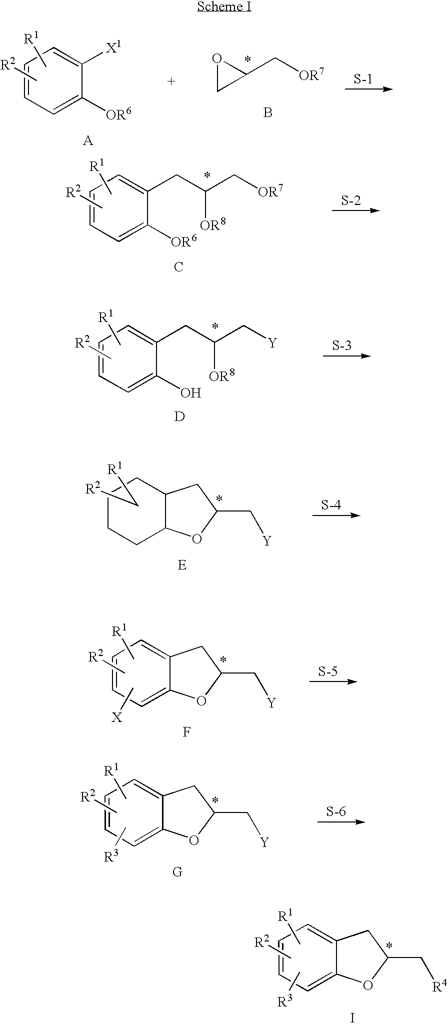

In Scheme I above, each of R¹, R², R³, R⁴, R⁶, R⁸, Y, X, and X¹ is as defined below and in classes and subclasses as described herein.

At step S-1, the conversion of a compound of formula A to compound of formula C, wherein R⁸ is hydrogen, is performed via a metal-halogen exchange reaction, followed by formation of an organocuprate. First, the compound of formula A is treated with a suitable Grignard reagent or an alkyl lithium then a chiral non-racemic epoxide of formula B: 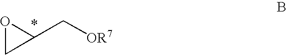
wherein R⁷ is a suitable hydroxyl protecting group. In other embodiments, said reagent is of formula RMgX², wherein X² is halogen and R is an alkyl group. In some embodiments, the organocuprate is formed utilizing CuBrSMe₂ or CuCN. In other embodiments the chiral non-racemic glycidyl ether is a glycidyl benzyl ether. One of ordinary skill in the art would recognize that the compound of formula C wherein R⁸ is hydrogen may be protected such that R⁸ is a hydroxyl protecting group.

At step S-2, the hydroxyl protecting group R⁶ of formula C is removed by suitable deprotection conditions. Deprotection conditions for removing hydroxyl protecting groups are known to one of ordinary skill in the art and include those described in detail in T. W. Greene and P. G. M. Wuts, “Protecting Groups in Organic Synthesis” (1991). A wide variety of techniques and reagents are available for the removal of hydroxyl protecting groups. Such techniques and agents are known to one skilled in the art. Hydroxyl protecting group can be removed, for example, by base hydrolysis, acid hydrolysis, or hydrogenation. In some embodiments, the removal of an hydroxyl protecting group is accomplished by acid hydrolysis. In some embodiments, the acid hydrolysis is performed in the presence of BBr₃ or a mixture of BBr₃ and BCl₃. In other embodiments, the removal of the protecting group is accomplished under basic conditions.

One of ordinary skill in the art would recognize that in certain embodiments, the R⁶ protecting group is removed under HBr/HOAc conditions, the R⁸ protecting group and Y group may be incorporated into the compound of formula D as acetyl and bromo, respectively.

The cyclization of a compound of formula D to a compound of formula E, as depicted at step S-3, is achieved by a variety of conditions. For example, when R⁸ is a base-labile hydroxyl protecting group, then the treatment of a compound of formula D can effect both deprotection of the R⁸ group and cyclization. Alternatively, the R⁸ protecting group may be removed prior to cyclization by conditions suitable for removing that group. Such conditions include reduction, treatment with acid, and the like as described in Greene. When the R⁸ protecting group is removed prior to cyclization such that a diol compound is formed, the cyclization of that compound to afford a compound of formula E may be achieved by dehydration. Such dehydration reactions are known to one of ordinary skill in the art and include Mitsunobu reactions.

As defined herein, the X group of formula F is halogen or triflate. The conversion of a compound of formula E to a compound of formula F wherein X is halogen is accomplished by halogenation reaction. One of ordinary skill in the art would recognize that a variety of halogenating agents are suitable for preparing a compound of formula F from a compound of formula E. In certain embodiments, X is bromo and the halogenating agent used at step S-4 is bromine. In other embodiments, X is bromo and the halogenating agent used at step S-4 is a compound containing an N—Br group (e.g., N-bromosuccinimide). Other brominating agents are known to those skilled in the art.

For preparing compounds of formula F wherein the X group is triflate, the compound of formula E is first formylated then the formyl group is converted to a hydroxyl group via Baeyer-Villiger procedure. The resulting hydroxyl group is then converted to a triflate group by ordinary methods.

At step S-5, the X group of formula F is coupled to the aryl or heteroaryl ring of R³ via Suzuki coupling reaction. Catalyst and reaction conditions for the Suzuki reaction of step S-5 above are well known in the art. See, for example, Miyaura, N.; Suzuki, A. Chem. Rev. 1995, 95, 2457. In certain embodiments, the Suzuki coupling at step S-5 is performed in the presence of a palladium containing compound. In other embodiments, the palladium containing compound is Pd(PPh₃)₄.

As defined herein, the Y group of formulae D, E, F, and G is a suitable leaving group. At step S-6, the Y group of formula G is displaced with a suitably protected amino group to form a compound of formula I wherein R⁴ is a protected amino group or an amino group of formula HN(R⁵)(R^{5a}). Alternatively, a compound of formula F is treated with an alkali metal azide to produce a compound of formula G wherein R⁴ is N₃.

Unless otherwise indicated, the following terms have the following meanings:

The term “alkyl,” as used herein, refers to a hydrocarbon group having 1 to 8 carbon atoms, preferably 1 to 6 carbon atoms, and more preferably 1 to 4 carbon atoms. The term “alkyl” includes, but is not limited to, straight and branched groups such as methyl, ethyl, n-propyl, isopropyl, n-butyl, isobutyl, sec-butyl, t-butyl, n-pentyl, isopentyl, neo-pentyl, n-hexyl, and isohexyl. The term “lower alkyl” refers to an alkyl group having 1 to 4 carbon atoms.

The term “alkenyl,” as used herein refers to a straight or branched hydrocarbon group having 2 to 8 carbon atoms and that contains 1 to 3 double bonds. Examples of alkenyl groups include vinyl, prop-1-enyl, allyl, methallyl, but-1-enyl, but-2-enyl, but-3-enyl, or 3,3-dimethylbut-1-enyl. The term “lower alkenyl” refers to a straight or branched alkenyl group having 1 to 4 carbon atoms.

The term “cycloaliphatic,” as used herein, refers to a saturated or partially unsaturated hydrocarbon monocyclic or bicyclic ring having 3 to 10 carbon atoms and more preferably 5 to 7 carbon atoms. In certain embodiments, the cyclic cycloaliphatic group is bridged. As used herein, the term “bridged” refers to a cycloaliphatic group that contains at least one carbon-carbon bond between two non-adjacent carbon atoms of the cycloalkyl ring. As used herein, the term “partially unsaturated” refers to a nonaromatic cycloaliphatic group containing at least one double bond and, in certain embodiments, only one double bond. In certain embodiments, the cycloaliphatic group is saturated. The cycloaliphatic group may be unsubstituted or substituted as described hereinafter.

The term “alkylcycloaliphatic,” as used herein, refers to the group —(CH₂)ᵣcycloaliphatic, where cycloaliphatic is as defined above and r is 1 to 6, preferably 1 to 4, and more preferably 1 to 3.

The term “heterocycloalkyl,” as used herein, refers to a 3 to 10 membered monocyclic or bicyclic ring having 1-3 heteroatoms independently selected from oxygen, nitrogen, or sulfur. In certain embodiments, heterocycloalkyl refers to a 5 to 7 membered ring having 1-2 heteroatoms independently selected from oxygen, nitrogen, or sulfur. The heterocycloalkyl group may be saturated or partially unsaturated, and may be monocyclic or bicyclic (such as bridged). Preferably, the heterocycloalkyl is monocyclic. The heterocycloalkyl group may be unsubstituted or substituted as described hereinafter.

The term “aryl” used alone or as part of a larger moiety as in “aralkyl”, “aralkoxy”, or “aryloxyalkyl”, refers to monocyclic, bicyclic and tricyclic ring systems having a total of six to fourteen ring members, wherein at least one ring in the system is aromatic and wherein each ring in the system contains 3 to 7 ring members. The term “aryl” may be used interchangeably with the term “aryl ring”. The term “aryloxy,” as used herein, refers to the group —OAr, where Ar is a 6-10 membered aryl group. The term “aralkoxy”, as used herein, refers to a group of the formula —O(CH₂)ᵣAr, wherein r is 1-6. The term “aryloxyalkyl”, as used herein, refers to a group of the formula —(CH₂)ᵣOAr, wherein r is 1-6.

The term “heteroaryl”, used alone or as part of a larger moiety as in “heteroaralkyl” or “heteroarylalkoxy”, refers to monocyclic, bicyclic and tricyclic ring systems having a total of five to fourteen ring members, wherein at least one ring in the system is aromatic, at least one ring in the system contains one or more heteroatoms independently selected from nitrogen, oxygen, or sulfur, and wherein each ring in the system contains 3 to 7 ring members. The term “heteroaryl” may be used interchangeably with the term “heteroaryl ring” or the term “heteroaromatic”. In certain embodiments, such heteroaryl ring systems include furanyl, thienyl, pyrazolyl, imidazolyl, isoxazolyl, oxadiazolyl, oxazolyl, pyrrolyl, pyridyl, pyrimidyl, pyridazinyl, triazinyl, thiazolyl, triazolyl, tetrazolyl, quinolinyl, isoquinolinyl, quinazolinyl, indolinyl, indazolyl, benzothienyl, benzofuranyl, benzisoxazolyl, benzimidazolyl, benzothiazolyl, benzoxazolyl, isoindolyl, and acridinyl, to name but a few. Any aryl, heteroaryl, cycloaliphatic or heterocycloalkyl may optionally be substituted with 1 to 5 substituents independently selected from halogen, hydroxyl, cyano, alkyl of 1 to 6 carbon atoms, perfluoroalkyl of 1 to 6 carbon atoms, alkoxy of 1 to 6 carbon atoms, or perfluoroalkoxy of 1 to 6 carbon atoms.

Any aryl, heteroaryl, cycloaliphatic, or heterocycloaliphatic group may optionally be substituted with 1 to 5 substituents independently selected from halogen, hydroxyl, C₁₋₆ alkyl, C₁₋₆ haloalkyl, O(C₁₋₆ alkyl), or O(C₁₋₆ haloalkyl).

The term “heteroaralkyl”, as used herein, refers to a group of the formula —(CH₂)ᵣHet, wherein Het is a heteroaryl group as defined above and r is 1-6. The term “heteroarylalkoxy”, as used herein, refers to a group of the formula —O(CH₂)ᵣHet wherein Het is a heteroaryl group as defined above and r is 1-6.

The term “perfluoroalkyl,” as used herein, refers to an alkyl group as defined herein in which all hydrogen atoms are replaced with fluorine.

The term “lower haloalkyl”, as used herein, refers to a C₁₋₄ alkyl group as defined herein in which one or more hydrogen atoms are replaced with a halogen atom.

The term “alkanesulfonamido,” as used herein, refers to the group R—S(O)₂—NH— where R is an alkyl group of 1 to 6 carbon atoms.

The term “alkoxy,” as used herein, refers to the group R—O— where R is an alkyl group of 1 to 6 carbon atoms.

The term “perfluoroalkoxy,” as used herein, refers to the group R—O where R is a perfluoroalkyl group of 1 to 6 carbon atoms.

The terms “monoalkylamino” and “dialkylamino,” as used herein, respectively refer to —NHR and —NR^{a}R^{b}, where R, R^{a} and R^{b} are each an independently selected C₁₋₆ alkyl group.

The terms “halogen” or “halo,” as used herein, refer to chlorine, bromine, fluorine or iodine.

The term “protecting group” such as “hydroxyl protecting group” and “amine protecting group” are well understood by one skilled in the art. In particular one skilled in the art is aware of various protecting groups for use to protect hydroxyl and primary and secondary amine groups. Protecting groups, including include those described for example, in T. W. Greene and P. G. M. Wuts, “Protecting Groups in Organic Synthesis” (1991) provided that they are suitable for use in the chemistries described herein. Particular examples of hydroxyl protecting groups include methyl, benzyl, benzyloxymethyl, or allyl.

Amino protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable amino protecting groups, taken with the —NH— moiety to which it is attached, include, but are not limited to, aralkylamines, carbamates, allyl amines, amides, and the like. Examples of such groups include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, benzyl (Bn), fluorenylmethylcarbonyl (Fmoc), acetyl, chloroacetyl, dichloroacetyl, trichloroacetyl, phenylacetyl, trifluoroacetyl, benzoyl, and the like. In other embodiments, an amino protecting group is acetyl, chloroacetyl, dichloroacetyl, trichloroacetyl, phenylacetyl, or trifluoroacetyl. In still other embodiments, an amino protecting group is phthalimide or azide.

Suitable leaving groups are well known in the art, e.g., see, “Advanced Organic Chemistry,” Jerry March, 5^{th} Ed., pp. 445-448, John Wiley and Sons, N.Y. Such leaving groups include, but are not limited to, halogen, alkoxy, sulphonyloxy, optionally substituted alkylsulphonyloxy, optionally substituted alkenylsulfonyloxy, optionally substituted arylsulfonyloxy. Examples of suitable leaving groups include chloro, iodo, bromo, fluoro, methanesulfonyl(mesyl),tosyl, triflate, nitrophenylsulfonyl(nosyl), bromophenylsulfonyl(brosyl), and the like.

Halogenating agents are those agents known in the art of organic synthesis to be capable of donating a halogen to an aromatic system. Examples of halogenating agents include, but are not limited to halophosphorous (such as phosphorous triiodide, phosphorous tribromide or phosphorous pentachloride), N-halosuccinimide, and thionyl halide (such as thionyl chloride).

The Baeyer-Villiger reaction or procedure is well known to those skilled in the art. This reaction is commonly used to covert aryl aldehydes for ketones to phenols via hydrolysis of the intermediate esters. See, for example, Jerry March, Advanced Organic Chemistry, 1992, 4^{th} Ed., p. 1098. The oxidation utilizes a peracid reagent.

The Suzuki coupling reaction is well known to those skilled in the art. In this reaction, a boronic acid and an aryl halide or triflate are coupled via a catalyzed process. Typical catalysts include palladium catalysts.

The compounds of the present invention may contain an asymmetric atom, and some of the compounds may contain one or more asymmetric atoms or centers, which may thus give rise to optical isomers (enantiomers) and diastereomers. In certain embodiments, the asymmetric atom is indicated with a “*”. When shown without respect to the stereochemistry, the present invention includes all optical isomers (enantiomers) and diastereomers (geometric isomers); as well as the racemic and resolved, enantiomerically pure R and S stereoisomers; as well as other mixtures of the R and S stereoisomers and pharmaceutically acceptable salts thereof. Optical isomers may be obtained in pure form by standard procedures known to those skilled in the art, and include, but are not limited to, diastereomeric salt formation, kinetic resolution, and asymmetric synthesis. It is also understood that this invention encompasses all possible isomers, and mixtures thereof, which may be obtained in pure form by standard separation procedures known to those skilled in the art, and include, but are not limited to, column chromatography, thin-layer chromatography, and high-performance liquid chromatography. Thus, the compounds of this invention include racemates, enantiomers, or geometric isomers of the compounds shown herein.

It is recognized that atropisomers of the present compounds may exit. The present invention thus encompasses atropisomeric forms of compounds of formula I and II, as defined above, and in classes and subclasses described above and herein. For definitions and an extensive discourse on atropisomers, see: Eliel, E. L. Stereochemistry of Organic Compounds (John Wiley & Sons, 1994, p 1142), which is incorporated herein by reference in its entirety.

The term “pharmaceutically acceptable salts” or “pharmaceutically acceptable salt” refers to salts derived from treating a compound of formula I with an organic or inorganic acid such as, for example, acetic, lactic, citric, cinnamic, tartaric, succinic, fumaric, maleic, malonic, mandelic, malic, oxalic, propionic, hydrochloric, hydrobromic, phosphoric, nitric, sulfuric, glycolic, pyruvic, methanesulfonic, ethanesulfonic, toluenesulfonic, salicylic, benzoic, or similarly known acceptable acids. In certain embodiments, the present invention provides the hydrochloride salt of a compound of formula I.

In some embodiments, certain reactions of the present invention are stereoselective. In other embodiments, certain reactions of the present invention are stereospecific.

The term “stereospecific” as used herein, is meant a reaction where starting materials differing only in their spacial configuration are converted to stereoisomerically distinct products. For example, in a stereospecific reaction, if the starting material is enantiopure (100% enantiomer excess “ee”), the final product will also be enantiopure. Similarly if the starting material has an enantiomer excess of about 50%, the final product will also have about a 50% enantiomer excess.

By “stereoselective” as used herein, it is meant a reaction where one stereoisomer is preferentially formed over another. Preferably, the process of the present invention will produce a dihydrobenzofuran having an enantiomer excess of at least about 30%, more preferably at least about 40%, and most preferably at least about 50%, where enantiomer excess is the mole percent excess of a single enantiomer over the racemate.

“Enantiomer excess” or “% ee” as used herein refers to the mole percent excess of a single enantiomer over the racemate.

As used herein, the term “chiral non-racemic” is used interchangeably with “enantiomerically enriched” and signifies that one enantiomer makes up more than 50% of the preparation. In certain embodiments, the term enantiomerically enriched signifies that at least 60% of the preparation is one of the enantiomers. In other embodiments, the term signifies that at least 75% of the preparation is one of the enantiomers. In other embodiments, the term signifies that at least 95% of the preparation is one of the enantiomers. is meant a nonracemic mixture of chiral molecules. In some embodiments, the chiral non-racemic compounds have more than about 30% ee. In other embodiments, the compounds have more than about 50% ee, or more than about 80% ee, or more than about 90% ee, or more than 95% ee, or more than 99% ee.

The process of the present invention preferably produces dihydrobenzofuran derivatives having an enantiomer excess of at least about 30%, more preferably at least about 50%, and most preferably at least about 95%.

“Organic impurities” as used herein, refers to any organic by-product or residual material present in the desired dihydrobenzofuran product, and do not include residual solvents or water. “Total organic impurities” refer to the total amount of organic impurities present in the desired dihydrobenzofuran product. Percent organic impurities such as total organic impurities and single largest impurity, unless otherwise stated are expressed herein as HPLC area percent relative to the total area of the HPLC chromatogram. The HPLC area percent is reported at a wavelength where the desired product and maximum number of organic impurities absorb.

According to one aspect, the present invention provides a method for preparing an enantiomerically enriched compound of formula II: 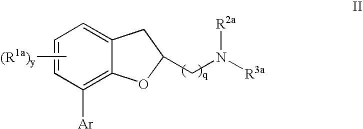
or a pharmaceutically acceptable salt thereof, wherein:
q is one or two;
each of R^{2a} and R^{3a} is independently hydrogen, methyl, ethyl, 2-fluoroethyl, 2,2-difluoroethyl or cyclopropyl;
each R^{1a} is independently hydrogen, halogen, OH, lower alkyl, lower alkoxy, lower haloalkyl, lower haloalkoxy, or CN;
Ar is thienyl, furyl, pyridyl, or phenyl, wherein Ar is optionally substituted with one or more R^{x} subsituents;
each R^{x} is independently selected from halogen, OH, lower alkyl, lower alkoxy, lower haloalkyl, lower haloalkoxy, or CN; and
y is 0, 1, 2, or 3.

As defined generally above, the Ar group of formula II is thienyl, furyl, pyridyl, or phenyl, wherein Ar is optionally substituted with one or more subsituents independently selected from halogen, OH, lower alkyl, lower alkoxy, haloalkyl, haloalkoxy, or CN. In certain embodiments, the Ar group of formula II is unsubstituted phenyl. In other embodiments, the Ar group of formula II is phenyl with at least one substituent in the ortho position. In other embodiments, the Ar group of formula II is phenyl with at least one substituent in the ortho position selected from halogen, lower alkyl, lower alkoxy, or trifluoromethyl. According to another aspect the present invention provides a compound of formula II wherein Ar is phenyl di-substituted in the ortho and meta positions with independently selected halogen, lower alkyl, or lower alkoxy. Yet another aspect of the present invention provides a compound of formula II wherein Ar is phenyl di-subsituted in the ortho and para positions with independently selected halogen, lower alkyl, or lower alkoxy. In other embodiment, the present invention provides a compound of formula II wherein Ar is phenyl di-subsituted in the two ortho positions with independently selected halogen, lower alkyl, or lower alkoxy. Exemplary substituents on the phenyl moiety of the Ar group of formula II include OMe, fluoro, chloro, methyl, and trifluoromethyl.

In certain embodiments, the Ar group of formula II is selected from the following:

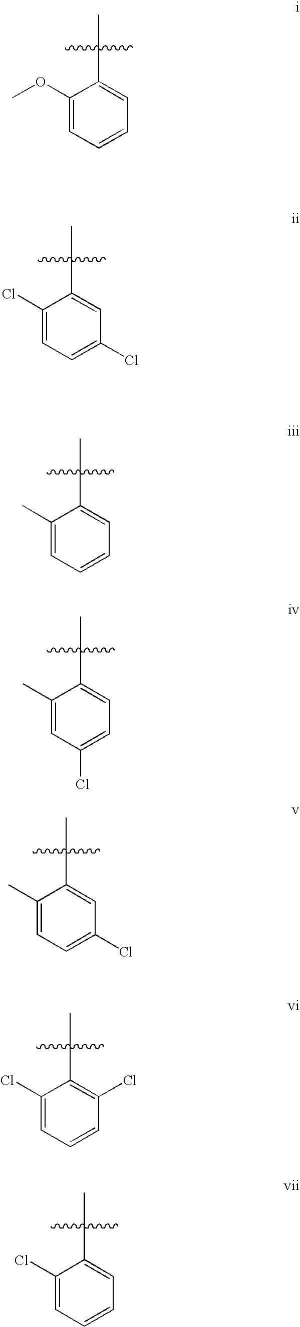

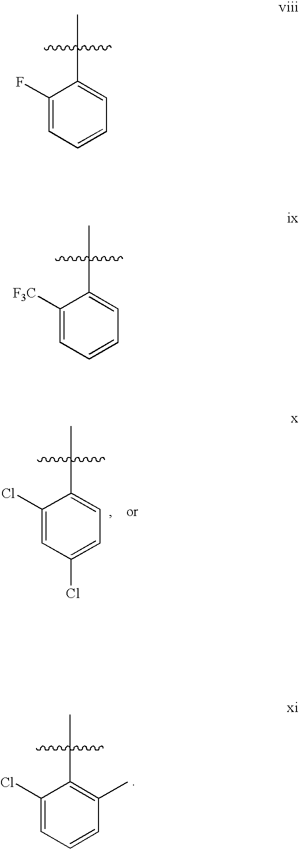

In certain embodiments, the present invention provides methods for preparing a compound of formula IIIa or IIIb: 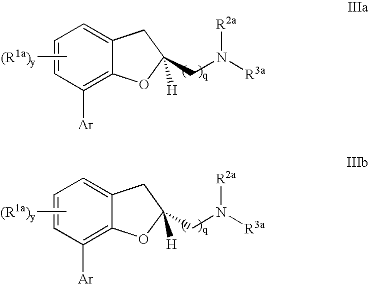
or a pharmaceutically acceptable salt thereof, wherein each R^{1a}, R^{2a}, R^{3a}, R^{x}, y, and q are as defined above for compounds of formula II and in classes and subclasses as described above and herein.

According to another embodiment, the present invention provides methods for preparing a compound of formula IIIc or IIId: 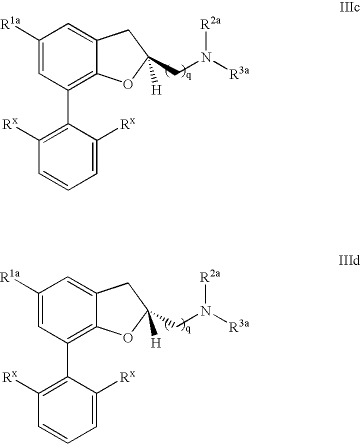
or a pharmaceutically acceptable salt thereof, wherein each of R^{1a}, R^{2a}, R^{3a}, R^{x}, y, and q is as defined above for compounds of formula H and in classes and subclasses as described above and herein.

The invention also concerns intermediates of the processes of the present invention.

In certain embodiments, the present invention provides a method for preparing a compound of formula I: 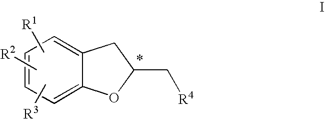
or a pharmaceutically acceptable salt thereof, wherein:
R¹ and R² are each independently hydrogen, chlorine, fluorine, CN, —OH, C₁₋₈ alkyl, C₁-₆ perfluoroalkyl, C₁-₆ alkoxy, C₁-₆ perfluoroalkoxy, 6-10 membered aryl, 6-10 membered aryloxy, 5-10 membered heteroaryl having 1 to 4 heteroatoms independently selected from nitrogen, oxygen or sulfur, C₂-₈ alkenyl, C₁-₆ alkanesulfonamido, dialkylamino of 1 to 6 carbon atoms per alkyl moiety, C₃-₈ cycloaliphatic, or 3-8 membered heterocycloalkyl having 1 to 3 heteroatoms independently selected from nitrogen, oxygen or sulfur; or R¹ and R² when adjacent to each other may be taken together with the carbon atoms to which they are attached to form a cyclic moiety selected from a monocyclic cycloaliphatic of 3 to 8 carbon atoms, a bridged cycloaliphatic of 5 to 10 carbon atoms, a 3 to 8 membered heterocycloaliphatic having 1 to 3 heteroatoms each independently selected from nitrogen, oxygen, or sulfur, 6-10 membered aryl, or a 5-10 membered heteroaryl having 1 to 3 heteroatoms each independently selected from nitrogen, oxygen, or sulfur, wherein the monocyclic cycloaliphatic or the heterocycloaliphatic may be optionally substituted at a single carbon atom with a 3-5 membered cycloalkyl ring or a 3-5 membered heterocycloalkyl ring having 1-2 heteroatoms independently selected from nitrogen, oxygen, or sulfur, to form a spirocyclic group;
R³ is hydrogen, 6-10 membered aryl, or 5-10 membered heteroaryl having 1 to 4 heteroatoms independently selected from nitrogen, oxygen or sulfur, wherein R³ is optionally substituted with one or more R^{x} groups;
each R^{x} is independently selected from halogen, OH, lower alkyl, lower alkoxy, lower haloalkyl, lower haloalkoxy, or CN;
R⁴ is CN, N₃, or N(R⁵)(R^{5a}); and
R⁵ and R^{5a} are each independently hydrogen, an amine protecting group, C₁₋₆ alkyl, lower haloalkyl, 3-6 membered cycloaliphatic, or alkylcycloaliphatic, or R⁵ and R^{5a} are taken together with the nitrogen to which they are attached to form a cyclic amine protecting group or a 3-6 membered saturated or partially unsaturated ring having 1-2 heteroatoms independently selected from nitrogen, oxygen, or sulfur,
   wherein said method comprises one or more of the steps depicted in Scheme I above. In certain embodiments, said method comprises all of the steps depicted in Scheme I above.

In certain embodiments, at least one of the R¹, R², and R³ groups of formula I is 6-10 membered aryl, or 5-10 membered heteroaryl having 1 to 4 heteroatoms independently selected from nitrogen, oxygen or sulfur. In certain other embodiments, R¹ and R² are adjacent to each other and may be taken together with the carbon atoms to which they are attached to form a cyclic moiety selected from a monocyclic cycloaliphatic of 3 to 8 carbon atoms, a bridged cycloaliphatic of 5 to 10 carbon atoms, a 3 to 8 membered heterocycloaliphatic having 1 to 3 heteroatoms each independently selected from nitrogen, oxygen, or sulfur, 6-10 membered aryl, or a 5-10 membered heteroaryl having 1 to 3 heteroatoms each independently selected from nitrogen, oxygen, or sulfur, wherein the monocyclic cycloaliphatic or the heterocycloaliphatic may be optionally substituted at a single carbon atom with a 3-5 membered cycloalkyl ring or a 3-5 membered heterocycloalkyl ring having 1-2 heteroatoms independently selected from nitrogen, oxygen, or sulfur, to form a spirocyclic group.

According to another embodiment, the present invention provides a method for preparing a compound of formula I-a: 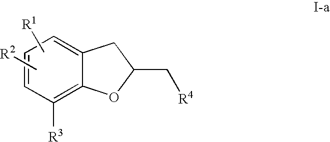
or a pharmaceutically acceptable salt thereof, wherein:
R¹ and R² are each independently hydrogen, chlorine, fluorine, CN, —OH, C₁₋₈ alkyl, C₁-₆ perfluoroalkyl, C₁-₆ alkoxy, C₁-₆ perfluoroalkoxy, 6-10 membered aryl, 6-10 membered aryloxy, 5-10 membered heteroaryl having 1 to 4 heteroatoms independently selected from nitrogen, oxygen or sulfur, C₂-₈ alkenyl, C₁-₆ alkanesulfonamido, dialkylamino of 1 to 6 carbon atoms per alkyl moiety, C₃-₈ cycloaliphatic, or 3-8 membered heterocycloalkyl having 1 to 3 heteroatoms independently selected from nitrogen, oxygen or sulfur; or R¹ and R² when adjacent to each other may be taken together with the carbon atoms to which they are attached to form a cyclic moiety selected from a monocyclic cycloaliphatic of 3 to 8 carbon atoms, a bridged cycloaliphatic of 5 to 10 carbon atoms, a 3 to 8 membered heterocycloaliphatic having 1 to 3 heteroatoms each independently selected from nitrogen, oxygen, or sulfur, 6-10 membered aryl, or a 5-10 membered heteroaryl having 1 to 3 heteroatoms each independently selected from nitrogen, oxygen, or sulfur, wherein the monocyclic cycloaliphatic or the heterocycloaliphatic may be optionally substituted at a single carbon atom with a 3-5 membered cycloalkyl ring or a 3-5 membered heterocycloalkyl ring having 1-2 heteroatoms independently selected from nitrogen, oxygen, or sulfur, to form a spirocyclic group;
R³ is hydrogen, 6-10 membered aryl, or 5-10 membered heteroaryl having 1 to 4 heteroatoms independently selected from nitrogen, oxygen or sulfur, wherein R³ is optionally substituted with one or more R^{x} groups;
each R^{x} is independently selected from halogen, OH, lower alkyl, lower alkoxy, lower haloalkyl, lower haloalkoxy, or CN;
R⁴ is CN, N₃, or N(R⁵)(R^{5a}); and
R⁵ and R^{5a} are each independently hydrogen, an amine protecting group, C₁₋₆ alkyl, lower haloalkyl, 3-6 membered cycloaliphatic, or alkylcycloaliphatic, or R⁵ and R^{5a} are taken together with the nitrogen to which they are attached to form a cyclic amine protecting group or a 3-6 membered saturated or partially unsaturated ring having 1-2 heteroatoms independently selected from nitrogen, oxygen, or sulfur,
   wherein said method comprises one or more of the steps depicted in Scheme I above. In certain embodiments, said method comprises all of the steps depicted in Scheme I above.

According to another embodiment, the R³ group of formula I-a is selected from the following:

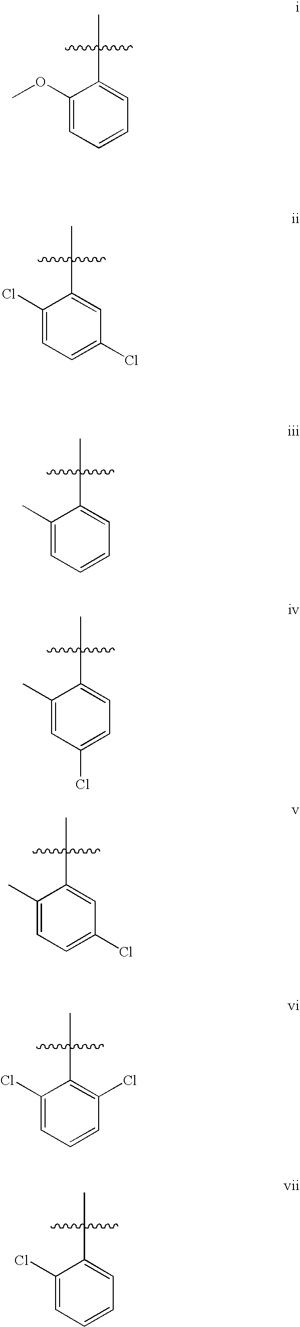

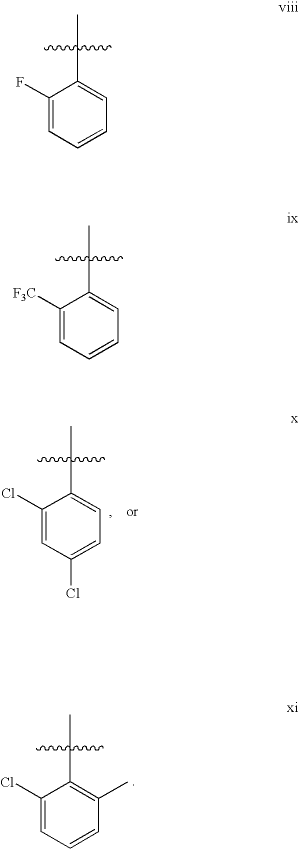

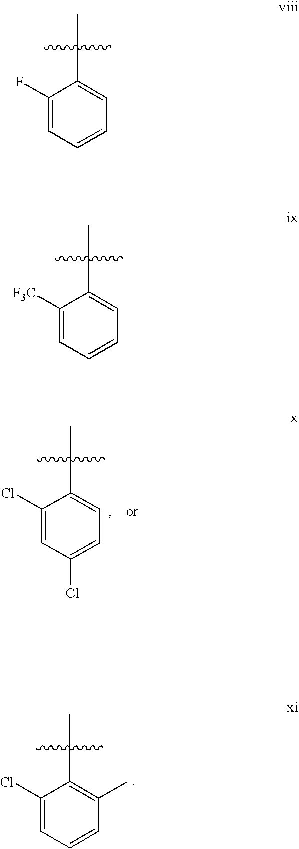

In certain embodiments, the present invention provides a method for preparing a compound of formula E: 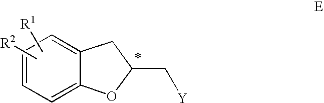
wherein:
R¹ and R² are each independently hydrogen, chlorine, fluorine, CN, —OH, C₁₋₈ alkyl, C₁-₆ perfluoroalkyl, C₁-₆ alkoxy, C₁-₆ perfluoroalkoxy, 6-10 membered aryl, 6-10 membered aryloxy, 5-10 membered heteroaryl having 1 to 4 heteroatoms independently selected from nitrogen, oxygen or sulfur, C₂-₈ alkenyl, C₁-₆ alkanesulfonamido, dialkylamino of 1 to 6 carbon atoms per alkyl moiety, C₃-₈ cycloaliphatic, or 3-8 membered heterocycloalkyl having 1 to 3 heteroatoms independently selected from nitrogen, oxygen or sulfur; or R¹ and R² when adjacent to each other may be taken together with the carbon atoms to which they are attached to form a cyclic moiety selected from a monocyclic cycloaliphatic of 3 to 8 carbon atoms, a bridged cycloaliphatic of 5 to 10 carbon atoms, a 3 to 8 membered heterocycloaliphatic having 1 to 3 heteroatoms each independently selected from nitrogen, oxygen, or sulfur, 6-10 membered aryl, or a 5-10 membered heteroaryl having 1 to 3 heteroatoms each independently selected from nitrogen, oxygen, or sulfur, wherein the monocyclic cycloaliphatic or the heterocycloaliphatic may be optionally substituted at a single carbon atom with a 3-5 membered cycloalkyl ring or a 3-5 membered heterocycloalkyl ring having 1-2 heteroatoms independently selected from nitrogen, oxygen, or sulfur, to form a spirocyclic group; and
Y is Br, Cl, or I,
   comprising the steps of:
   (a) providing a chiral non-racemic compound of formula D: 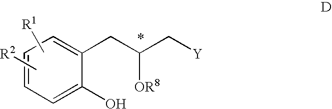
   wherein:
R¹ and R² are each independently hydrogen, chlorine, fluorine, CN, —OH, C₁₋₈ alkyl, C₁-₆ perfluoroalkyl, C₁-₆ alkoxy, C₁-₆ perfluoroalkoxy, 6-10 membered aryl, 6-10 membered aryloxy, 5-10 membered heteroaryl having 1 to 4 heteroatoms independently selected from nitrogen, oxygen or sulfur, C₂-₈ alkenyl, C₁-₆ alkanesulfonamido, dialkylamino of 1 to 6 carbon atoms per alkyl moiety, C₃-₈ cycloaliphatic, or 3-8 membered heterocycloalkyl having 1 to 3 heteroatoms independently selected from nitrogen, oxygen or sulfur; or R¹ and R² when adjacent to each other may be taken together with the carbon atoms to which they are attached to form a cyclic moiety selected from a monocyclic cycloaliphatic of 3 to 8 carbon atoms, a bridged cycloaliphatic of 5 to 10 carbon atoms, a 3 to 8 membered heterocycloaliphatic having 1 to 3 heteroatoms each independently selected from nitrogen, oxygen, or sulfur, 6-10 membered aryl, or a 5-10 membered heteroaryl having 1 to 3 heteroatoms each independently selected from nitrogen, oxygen, or sulfur, wherein the monocyclic cycloaliphatic or the heterocycloaliphatic may be optionally substituted at a single carbon atom with a 3-5 membered cycloalkyl ring or a 3-5 membered heterocycloalkyl ring having 1-2 heteroatoms independently selected from nitrogen, oxygen, or sulfur, to form a spirocyclic group;
Y is Br, Cl, or I; and
R⁸ is hydrogen or a suitable hydroxyl protecting group, and
   (b) cyclizing said compound of formula D to form a compound of formula E.

In some embodiments, the cyclization reaction is accomplished using a stereospecific dehydration reaction such as a dehydration reaction with Mitsunobu reaction conditions.

In certain embodiments, the process further comprises converting the compound of formula E to a compound of formula F: 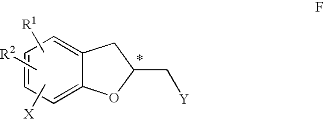
wherein R¹, R², and Y are as defined above and X is halogen or triflate.

In some aspects, the invention concerns the preparation of the compound of formula F, wherein X is halogen, by a process which comprises: contacting a compound of formula E: 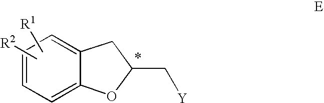
wherein R¹, R² and Y are as defined above,
   with a halogenating agent.

In other aspects, the present invention provides a method for preparing a compound of formula F, wherein X is triflate, said method comprising the steps of:
(a)formylating the compound of formula E to provide a formyl group,
(b) converting the formyl group to a hydroxyl group via Baeyer-Villiger conditions, and
(c) triflating the resulting hydroxyl group.

In certain embodiments, step (c) is performed with trifluoromethanesulfonic anhydride in the presence of a tertiary amine.

In some embodiments, the compound of formula D is produced by providing a compound of formula C′ 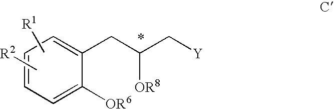
wherein R¹, R², R⁸ and Y are as defined above and R⁶ is a suitable hydroxyl protecting group,
   and removing the R⁶ protecting group from the compound of formula C′.

In certain aspects, the invention further comprises converting the compound of formula F: 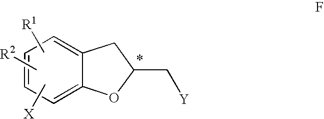
wherein R¹, R², X, and Y are as defined above,
   to a compound of formula I: 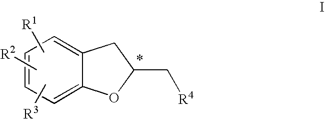
wherein R¹ and R² are as defined above;
R³ is 6-10 membered aryl, or 5-10 membered heteroaryl having 1 to 4 heteroatoms independently selected from nitrogen, oxygen or sulfur, wherein R³ is optionally substituted with one or more R^{x} groups;
each R^{x} is independently selected from halogen, OH, lower alkyl, lower alkoxy, lower haloalkyl, lower haloalkoxy, or CN;
R⁴ is CN, N₃, or N(R⁵)(R^{5a}); and
R⁵ and R^{5a} are each independently hydrogen, an amine protecting group, C₁₋₆ alkyl, lower haloalkyl, 3-6 membered cycloaliphatic, or alkylcycloaliphatic, or R⁵ and R^{5a} are taken together with the nitrogen to which they are attached to form a cyclic amine protecting group or a 3-6 membered saturated or partially unsaturated ring having 1-2 heteroatoms independently selected from nitrogen, oxygen, or sulfur.

In some embodiments of the invention, the conversion of the compound of formula D to the compound of formula E comprises the steps of:
(a) removing the R⁸ hydroxyl protecting group from the compound of formula D to produce a compound of the formula D-1: 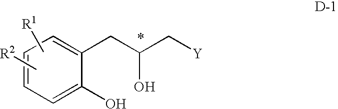
wherein R¹, R², R⁸ and Y are as defined above,
   and
   (b) stereospecifically cyclizing the compound of formula D-1 to produce a compound of formula E: 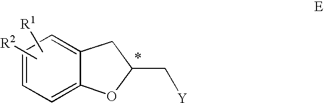
wherein R¹, R¹ and Y are as defined above.

In some aspects, the present invention provides a method for converting a compound of formula F: 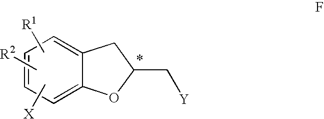
wherein R¹, R², X, and Y are as defined above,
   to a compound of formula I: 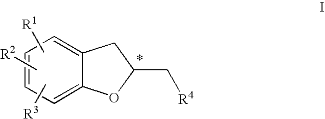
wherein R¹, R², R³, and R⁴ are as defined above;
   wherein said method comprises the steps of:
   (a) converting the compound of formula F to a compound of formula G: 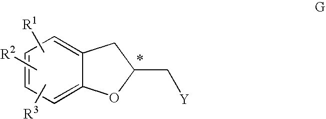
wherein R¹, R², R³, and Y are as defined above;
   and
   (b) reacting the compound of formula G with an amine or protected amine to produce a compound of formula I.

In yet other aspects, the compound of formula F is converted to a compound of formula G via a Suzuki coupling reaction.

In some aspects, the invention concerns processes where the compound of formula F is converted to a compound of formula I by a process which comprises the steps of:
(a) converting the compound of formula F to a compound of formula G: 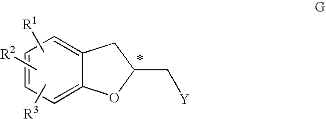
wherein R¹, R², R³, and Y are as defined above;
   (b) reacting the compound of formula G with an alkali metal azide such as sodium azide to produce a compound of formula G-1: 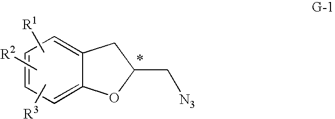
wherein R¹, R², and R³ are as defined above;
   and
   (c) reducing the compound of formula G-1 to produce a compound of formula I where R⁴ is NH₂.

In certain aspects, the present invention provides a method for preparing a compound of formula D: 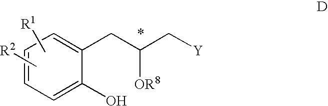
wherein each of R¹, R², R⁸, and Y is as defined above;
   comprising the steps of:
   (a) providing a compound of formula A: 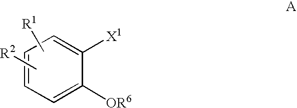
wherein each of R¹, R², R⁶, and X¹ is as defined above;
   (b) treating the compound of formula A with an chiral non-racemic compound of formula B: 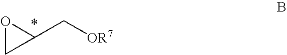
wherein R⁷ is a suitable hydroxyl protecting group;
   to form a compound of formula C: 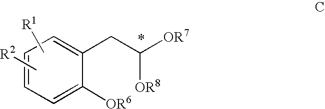
wherein R¹, R², R⁶, and R⁸ are as defined above and R⁷ is an acid-labile hydroxyl protecting group;
   and
   (c) reacting the compound of formula C with a hydrogen halide to produce a compound of formula D-1.

In some embodiments, the conversion of the compound of formula A to the compound of formula D comprises the steps of:
(a) treating a compound of the formula A with an chiral non-racemic compound of formula B: 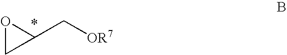
wherein R⁷ is a suitable hydroxyl protecting group;
   to form a compound of formula C-1: 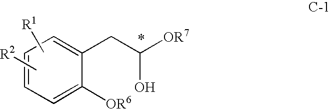
wherein R¹, R², and R⁶ are as defined above and R7 is a hydroxyl protecting group;
   and
   (b) converting the compound C-1 to a compound of formula D.

In certain embodiments, the conversion of compound A to compound C-1 comprises a metal-halogen exchange reaction, followed by formation of an organocuprate. The organocuprate is preferably reacted with an chiral non-racemic glycidyl ether to form C-1. In certain embodiments, the metal-halogen exchange reaction utilizes at least one of n-butyl lithium and iso-propyl magnesium chloride. In some embodiments, the organocuprate is formed utilizing CuBrSMe₂ or CuCN. In other embodiments the chiral non-racemic glycidyl ether is a glycidyl benzyl ether.

According to another embodiment, the present invention provides a method for preparing a compound of formula I: 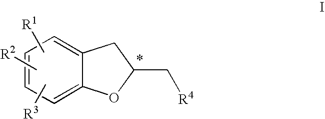
wherein each of R¹, R², R³, and R⁴ are as defined above;
   comprising the steps of:
   (a) providing a compound of formula D: 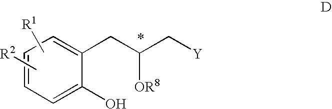
wherein R¹ and R² are as defined above, Y is Br and R⁸ is hydrogen or a base-labile hydroxyl protecting group;
   (b) converting the compound of formula D to a compound of the formula F-1: 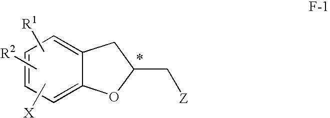
wherein R¹, R² and X are as defined above and Z is a suitable leaving group;
   and
   (c) converting the compound of formula F-1 to a compound of formula I.

In certain embodiments, the Z group of formula F-1 is an arylsulfonyl, alkylsulfonyl or halogen.

In certain embodiments, the conversion of the compound of formula D to the compound of formula F-1 comprises the steps of:
(a) cyclizing the compound of formula D (where R⁸ is H or a base-labile hydroxyl protecting group) by reacting with base to produce a compound of the formula: 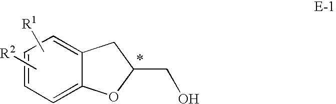
wherein R¹ and R² are as defined above;
   (b) converting the hydroxyl group of the compound of formula E-1 to a leaving group to provide a compound of the formula E-2: 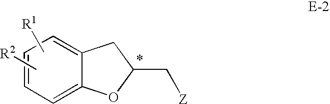
wherein R¹ and R² are as defined above and Z is a suitable leaving group;
   and
   (c) converting the compound of formula E-2 to a compound of formula F-1.

In some aspects of the invention, the conversion of the compound of formula E-2 to a compound of formula F-1 comprises either of the steps of: (a) formylating the compound of formula E-2 to provide a formyl group, converting the formyl group to a hydroxyl group via a Baeyer-Villiger procedure, and triflating the resulting hydroxyl group with trifluoromethanesulfonic anhydride in the presence of a tertiary amine to form a compound of formula F-1 wherein X is triflate, or (b) contacting a compound of formula E-2 with a halogenating agent to form a compound of formula F-1 wherein X is halogen.

In certain embodiments, the conversion of the compound of formula F-1 to the compound of formula I comprises the steps of:
(a) converting the compound of formula F-1 to a compound of formula G-1: 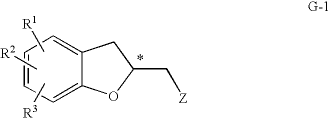
wherein R¹, R², R³, and Z are as defined above;
   and
   (b) converting the compound of formula G-1 to a compound of formula I.

In certain embodiments, the compound of formula F-1 is converted to a compound of formula G-1 by a Suzuki coupling reaction. In certain aspects, the conversion of the compound of formula G-1 to a compound of formula I comprises contacting the compound of formula G-1 with an amine or with sodium azide followed by reduction.

In some embodiments, the present invention provides a method for preparing a compound of formula D-1: 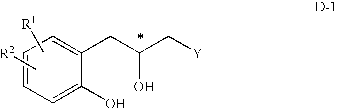
wherein R¹, R², R⁸ and Y are as defined above,
   comprising the steps of:
   (a) providing a compound of formula A: 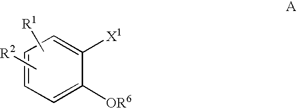
wherein R¹, R², R⁶ and X¹ are as defined above,
   (b) converting a compound of formula A to a compound of formula C: 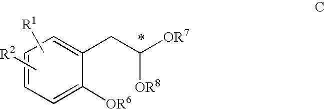
wherein R¹, R², R⁶, R⁷, and R⁸ are as defined above,
   (c) reacting the compound of formula C with a hydrogen bromide to produce a compound of formula C′: 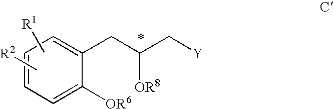
wherein R¹, R², R⁸, and R⁶ are as defined above and Y is Br, and
   (d) if R⁶ group of formula C′ is a hydroxyl protecting group, then further comprising the step of removing the protecting group to produce a compound of formula D where Y is Br.

In other aspects, the invention concerns products of the processes of the invention.

### General Synthetic Schemes

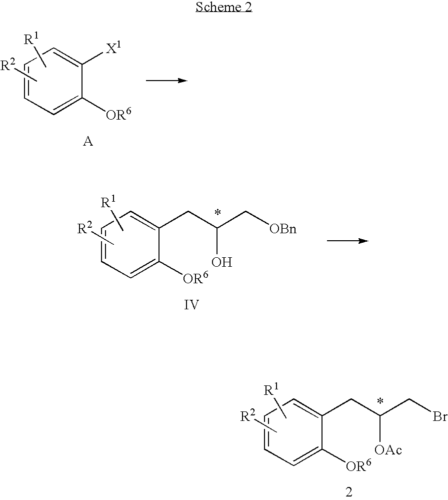

In Scheme 2 above, incorporation of the R⁶ protecting group of intermediate A may be accomplished using any hydroxyl protecting reagent known to those skilled in the art. Such reagents include, but are not limited to, iodomethane or benzyl bromide. In one embodiment, R⁶ is a methyl group. As defined generally herein, X¹ is a halogen atom. In some embodiments, X¹ is bromine or iodine. The X¹ is then converted into a chiral non-racemic derivative of formula IV. This conversion includes the step of metal-halogen exchange, using, for example, n-butyl lithium or isopropylmagnisium chloride, followed by forming an organocuprate, using, for example, CuBrSMe₂ or CuCN. The organocuprate intermediate is then reacted with an chiral non-racemic glycidyl ether of the formula 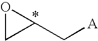
where A is a protected hydroxyl group and/or a leaving group to form IV. Preferred chiral non-racemic glycidyl ethers include chiral non-racemic glycidylbenzyl ether. In other embodiments, the glycidylbenzyl ether is the (+)-S-enantiomer. The chiral non-racemic compound of formula IV may then be further reacted to produce the bromine derivative 2. This reaction may be accomplished, for example, with a solution of 30% hydrogen bromide in acetic acid to provide intermediate 2.

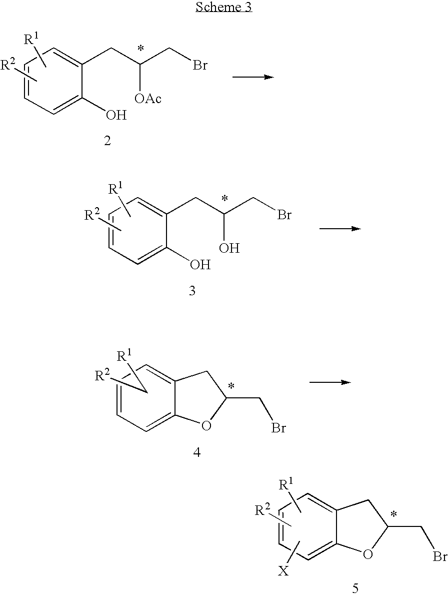

There are various ways to carry out the stereospecific cyclization reaction starting with intermediate 2. According to one embodiment, the cyclization is carried out using a stereospecific dehydration reaction, such as under Mitsunobu reaction conditions in the presence of triphenylphosphine and diethylazodicarboxylate. As shown in Scheme 3 above, acetoxy group of intermediate 2 can be deprotected according to conventional techniques to form compound 3. In some embodiments, this deprotection is accomplished under acidic condition. The cyclization reaction, Mitsunobu reaction in some embodiments, will stereospecifically convert 3 to intermediate 4. A halogen or trifluoromethanesulfonyloxy group (X) is then introduced to intermediate 4 by any suitable method known to those skilled in the art, such as bromination or iodination to form a compound 5 where X is Br or I. Alternatively, compound 4 is formylated followed by oxidation, hydrolysis and treatment with trifluoromethanesulfonic anhydride to generate triflate, to form intermediate 5 where X is triflate.

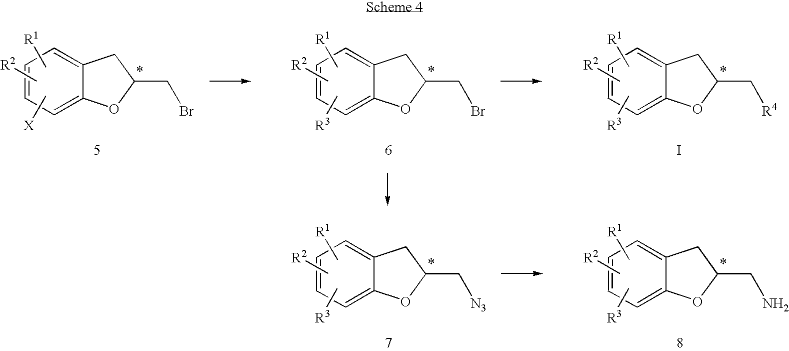

In Scheme 4 above, an aryl or heteroaryl R³ may be introduced to form a compound 6. This introduction is accomplished by the Suzuki coupling reaction. The bromine moiety of intermediate 6 may be displaced by different amines using conventional techniques to generate corresponding dihydrobenzofuran derivatives of formula I. The bromine in intermediate 6 may also be displaced by sodium azide using conventional techniques to form intermediate 7. Reduction of the azide is accomplished by any suitable method known to those skilled in the art forms the corresponding primary amine 8.

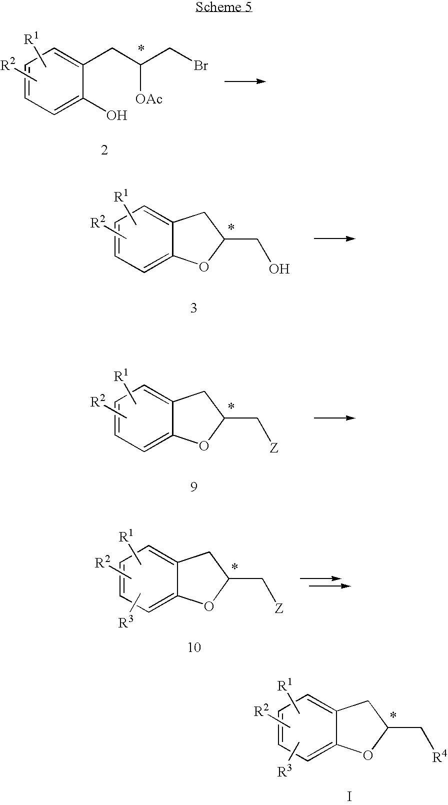

Cyclization of the bromide intermediate 2 to give 3 may be carried out in the presence of a suitable base that is, in some embodiments, an inorganic base such as an alkali metal or alkaline earth metal hydroxide or carbonate, such as potassium or sodium hydroxide or potassium carbonate. The reaction may be conducted in any suitable solvent. In some embodiments, the suitable solvent is a polar solvent, such as an alcoholic solvent (methanol or ethanol). In one embodiment, the cyclization reaction is carried out with aqueous sodium hydroxide in methanol to generate compound 3. The hydroxyl group of the compound of formula 3 may be converted to a leaving group such as arylsulfonyl, alkylsulfonyl or halogen. For example, compound 3 is treated with any arylsulfonyl chloride to form intermediate 9. A compound of formula I is then prepared according to Scheme 4 above.

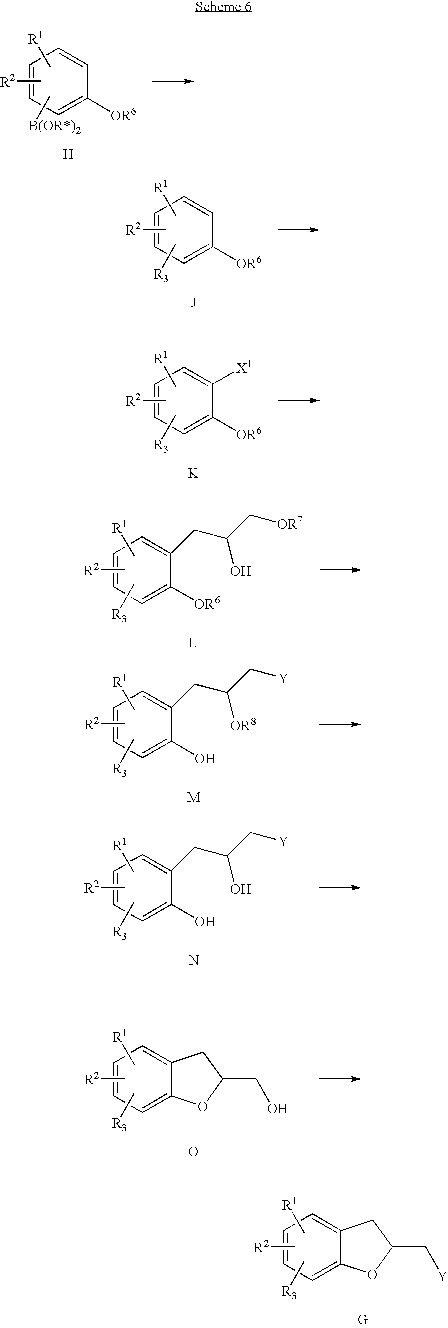

Scheme 6 above depicts an alternate method for preparing compounds of formula I or II in accordance with the present invention. As depicted in Scheme 6, the R³ moiety is incorporated to form a compound of formula J via Suzuki coupling. Specifically, a compound of formula H, wherein R* is hydrogen or a C₁₋₆ alkyl group, is treated with a compound of formula R³—OTf or R³Br in the presence of a palladium catalyst. The resulting compound of formula J is halogenated by methods known to one of ordinary skill in the art to form a compound of formula K wherein X¹ is halogen.

The conversion of a compound of formula K to a compound of formula G is performed in a manner substantially similar to that described herein for the conversion of a compound of formula A to a compound of formula G. Each of these steps is described in detail herein. One of ordinary skill in the art would recognize that the compound of formula G, prepared in accordance with Scheme 6, is readily transformed to a compound of formula I by the methods described herein.

### EXAMPLES

### Example 1

### (S)-1-Benzyloxy-3-(5-fluoro-2-methoxy-phenyl)propan-2-ol

To a solution of compound 4-fluoro-2-bromanisole (12.6 ml, 0.1 mol) in anhydrous tetrahydrofuran was added n-BuLi (2.5M in hexane, 39 ml, 0.1 mol)) at −78° C. The resulting mixture was stirred at −78° C. for a few hours until no more starting material was present. CuBrSMe₂ (10.0 g, 0.05 mol) was added to above mixture at −78° C., and the reaction temperature was slowly increased from −78° C. to −40° C. in 2 hours. Optical active glycidyl benzyl ether (3.71 ml, 0.025 mol) was introduced at −60° C., followed by BF₃OEt₂ (0.15 ml, 1.2 mmol). The reaction mixture was stirred at −60° C. to 10° C. in the overnight period. The solvent was removed under vacuum. Chromatography with 30% ethyl acetate in hexane afforded desired product 5.0 g (70%) as a clear oil. HRMS ESI m/e 308.1666 [M+NH4]⁺, Calc'd m/e 308.1662 [M+NH4]⁺; [α]=+8.1° (0.89%, MeOH).

### Example 2

### (S)-1-Benzyloxy-3-(5-chloro-2-methoxy-phenyl)propan-2-ol

To a solution of 4-chloro-2-bromanisole (21.5 g, 0.1 mol) in anhydrous tetrahydrofuran was added n-BuLi (2.5M in hexane, 38.8 ml, 0.1 mol)) at −78° C. The resulting mixture was stirred at −78° C. for a few hours until no more starting material present. CuBrSMe₂ (10.0 g, 0.05 mol) was added to above mixture at −78° C. once, the reaction temperature was slowly increased from −78° C. to −40° C. in 2 hours. Optical active glycidyl benzoether (3.71 ml, 0.025 mol) was introduced at −60° C., followed by BF₃OEt₂ (0.15 ml, 1.2 mmol). The reaction mixture was stirred at −60° C. to 10° C. in the overnight period. The solvent was removed under vacuum. Chromatography with 30% ethyl acetate in hexane afforded desired product 5.1 g (%) as a clear oil. HRMS ESI m/e 307.1096 [M+H]⁺, Calc'd 307.1101; [α]=+6.6° (1%, MeOH).

### Example 3

### (S)-1-Benzyloxy-3-(2-methoxy-5-methyl-phenyl)propan-2-ol

Starting from 2-bromo-4-methylanisole (14.05 ml, 0.1 mol) and following the procedure described for Example 1 gave the desired product 6.74 g (96%) as a clear oil.

HRMS EI m/e 286.1565 (M)+, Calc'd. 286.1569; [α]=15.67° (6.7 mg/0.7 ml, MeOH).

### Example 4

### (S)-1-Benzyloxy-3-(2-methoxy-phenyl)propan-2-ol

Starting from 2-bromoanisole (12.1 ml, 0.1 mol) and following the procedure described for Example 1 gave the desired product 5.4 g (82%) as a clear oil. HRMS EI m/e 272.1413 (M)+, Calc'd. 272.1412 [α]=+18.07° (c 5.5 mg/0.7 ml, MeOH).

### Example 5

### (S)-1-Benzyloxy-3-(2′,6′-dichloro-5-fluoro-2-methoxybiphenyl-3-yl)propan-2-ol

To a solution of 3-bromo-2′,6′-dichloro-5-fluoro-2-methoxy-biphenyl (2.2 g, 6.3 mmol) in anhydrous tetrahydrofuran was added i-PrMgCl (2.0 M in hexane, 3.45 ml, 6.9 mmol) at 0° C. The resulting mixture was stirred at 0° C. for hours until no more starting material present. A slurry of CuCN (0.28 g, 3.1 mmol) in THF was added to above mixture at −30° C. once, the mixture was stirred at −30° C. for 1 hour. Then (+)-(2S)-glycidyl benzylether (0.48 ml, 3.1 mmol) was introduced at −30° C. The reaction mixture was stirred at −30° C. to 10° C. in overnight period. The solvent was removed under vacuum. Chromatography with 30% ethyl acetate in hexane afforded desired product 1.28 g (94%) as a clear oil. HRMS ESI m/e 435.0946 [M−H]−, Calc'd 435.0930; [α]=+2.8° (c 5.7 mg/0.7 ml, DMSO).

### Example 6

### (S)-1-Benzyloxy-3-(6′-chloro-5,2′-difluoro-2-methoxybiphenyl-3-yl)propan-2-ol

Starting from 6′-chloro-5,2′-difluoro-2-methoxy-biphenyl (9.8 g, 29.3 mmol) and following the procedure described for Example 5 gave the desired product 7.4 g (60%) as a clear oil. MS ESI m/e 419.1 [M+H]⁺.

### Example 7

### (S)-Acetic acid 1-bromomethyl-2-(5-fluoro-2-hydroxy-phenyl)-ethyl ester

1-Benzyloxy-3-(5-fluoro-2-methoxy-phenyl)propan-2-ol (5.17 g, 17.8 mmol) was dissolved in 30% hydrogen bromide in acetic acid (40 ml). The reaction mixture was heated at 70° C. overnight. The solvent was removed under vacuum. The residue was dissolved in methylene chloride and washed with ammonium hydroxide. The organic solvent was removed under vacuum. Chromatography with 30% ethyl acetate in hexane afforded product 3.60 g (70%) as a light brown oil.

Elemental Analysis for: C₁₁H₁₂BrFO₃ Theory: C, 45.38 H, 4.15 Found: C, 45.24 H, 4.09

### Example 8

### (S)-Acetic acid 1-bromomethyl-2-(5-chloro-2-hydroxy-phenyl)-ethyl ester

Starting from 1-benzyloxy-3-(5-chloro-2-methoxy-phenyl)propan-2-ol (5.4 g, 17.6 mmol) and following the procedure described for Example 7 gave the desired product 3.8 g (70%) as a light brown oil. HRMS El m/e 305.9647 (M)+.

### Example 9

### (S)-Acetic acid 1-bromomethyl-2-(2-hydroxy-5-methyl-phenyl)-ethyl ester

Starting from 1-benzyloxy-3-(2-methoxy-5-methyl-phenyl)propan-2-ol (6.7 g, 23.3 mmol) and following the procedure described for Example 7 gave the desired product 6.24 g (93%) as a yellow oil. MS EI m/e 286 (M)+; [α]=−2.41° (c 5.8 mg/0.7 ml, MeOH)

### Example 10

### (S)-Acetic acid 1-bromomethyl-2-(2-hydroxy-phenyl)-ethyl ester

Starting from 1-benzyloxy-3-(2-methoxy-phenyl)propan-2-ol (5.40 g, 19.8 mmol) and following the procedure described for Example 7 gave the desired product 3.42 g (63%) as a yellow oil. [α]=−12.2° (c 1%, MeOH)

Elemental Analysis for: C₁₆H₁₅BrO₃ Theory: C, 48.37 H, 4.80 Found: C, 48.48 H, 4.78

### Example 11

### (S)-Acetic acid 3-(2-acetoxy-3-bromo-propyl)-2′,6′-dichloro-5-fluoro-biphenyl-2-yl ester

Starting from 1-benzyloxy-3-(2′,6′-dichlor-5-fluoro-2-methoxybiphenyl-3-yl)propan-2-ol (1.28 g, 2.9 mmol) and following the procedure described for Example 7 gave the desired product (1.12 g (80%)) as a light yellow oil. HRMS ESI m/e 476.9686 [M+H]+, Calc'd. 476.9671; [α]=+13.2° (c 1%, MeOH)

### Example 12

### (S)-Acetic acid 1-bromomethyl-2-(6′-chloro-5,2′-difluoro-2-hydroxy-biphenyl-3-yl)-ethyl ester

Starting from (S)-1-benzyloxy-3-(6′-chloro-5,2′-difluoro-2-methoxybiphenyl-3-yl)propan-2-ol (7.4 g, 17.7 mmol) and following the procedure described for Example 7 gave the desired product 2.72 g (37%) as a light yellow oil. MS EI m/e 418 M⁺; [α]=−7.4° (c 1%, MeOH)

### Example 13

### (S)-2-(3-Bromo-2-hydroxy-propyl)-4-fluoro-phenol

To a solution of acetic acid 1-bromomethyl-2-(5-fluoro-2-hydroxy-phenyl)-ether ester (3.57 g, 12.2 mmol) in methanol was added hydrogen chloride in ether (1.0 M, 49 ml, 48.8 mmol) at room temperature. The mixture was stirred at room temperature overnight. The solvent was removed under vacuum. Chromatography with 30% ethyl acetate afforded product 2.95 g (97%) as a clear oil. HRMS ESI m/e 246.9761 [M−H]+; Calc'd 246.9755. [α]=+8.2° (c 0.71%, MeOH)

### Example 14

### (S)-2-(3-Bromo-2-hydroxy-propyl)-4-chloro-phenol

Starting from acetic acid 1-bromomethyl-2-(5-chloro-2-hydroxy-phenyl)-ether ester (2.47 g, 3.2 mmol) and following the procedure described for Example 13 gave the desired product 1.68 g (79%) as a yellow oil. [α]=+9.8° (c 1%, MeOH), HRMS EI m/e 263.956 (M)+.

### Example 15

### (S)-2-(3-Bromo-2-hydroxy-propyl)-4-metyl-phenol

Starting from acetic acid 1-bromomethyl-2-(2-hydroxy-5-methyl-phenyl)-ether ester (6.24 g, 22 mmol) and following the procedure described for Example 13 gave the desired product 5.0 g (94%) as a clear oil. [α]=+13.8° (c 1%, MeOH), HRMS ESI m/e 243.0020 [M−H]−, Calc'd. 243.0021

### Example 16

### (S)-2-(3-Bromo-2-hydroxy-propyl)-phenol

Starting from acetic acid 1-bromomethyl-2-(2-hydroxy-phenyl)-ether ester (3.42 g, 12.5 mmol) and following the procedure described for Example 13 gave the desired product 2.71 g (93%) as a light yellow oil. MS ES m/e 229.0 [M−H]−; [α]=+16.46° (c 5.7 mg/0.7 ml, MeOH)

### Example 17

### (S)-3-(3-Bromo-2-hydroxy-propyl)-2′,6′,-dichloro-5-fluoro-biphenyl-2-ol

Starting from acetic acid 2-(2-acetoxy-2′,6′-dichloro-5-fluoro-biphenyl-3-yl)-1-bromomethyl-ethyl ester (1.6 g, 33.4 mmol) and following the procedure described for Example 13 gave the desired product 1.48 g (100%) as a light yellow oil. HRMS EI m/e 391.9391 (M)+, Calc'd. 391.9391; [α]=−4.76° (c 5.0 mg/0.7 ml, MeOH

### Example 18

### (S)-3-(3-Bromo-2-hydroxy-propyl)-2′-chloro-5,6′-difluorobiphenyl-2-ol

Starting from (S)-acetic acid 1-bromomethyl-2-(6′-chloro-5,2′-difluoro-2-hydroxy-biphenyl-3-yl)-ethyl ester (2.72 g, 6.5 mmol) and following the procedure described for Example 13 gave the desired product 2.2 g (90%) as a light yellow oil. MS EI m/e 376 (M)+.

### Example 19

### (R)-2-Bromomethyl-5-fluoro-2,3-dihydro-benzofuran

To a solution of 2-(3-bromo-2-hydroxy-propyl)-4-fluoro-phenol(1.97 g, 8 mmol) in tetrahydrofuran was added triphenyl phosphine (5.2 g, 20 mmol) and followed by DEAD (3.11 ml, 20 mmol) at room temperature. The reaction mixture was stirred at room temperature for 2 h. Solvent was removed under vacuum. Chromatography with 5% ethyl acetate afforded product 1.40 g (76%) as a clear oil. HRMS ESI m/e 228.9661 [M−H]−. [α]=−33.0° (c 1%, MeOH)

### Example 20

### (R)-2-Bromomethyl-5-methyl-2,3-dihydro-benzofuran

Starting from 2-(3-bromo-2-hydroxy-propyl)-4-metyl-phenol (5.0 g, 20 mmol) and following the procedure described for Example 19 gave the desired product 3.04 g (70 %) as a yellow oil. HRMS EI m/e 225.9998 (M)+; [α]=−41.13° (c 6.2/0.7 ml, MeOH)

### Example 21

### (R)-2-Bromomethyl-2,3-dihydro-benzofuran

Starting from 2-(3-bromo-2-hydroxy-propyl)-phenol (2.71 g, 12 mmol) and following the procedure described for Example 19 gave the desired product 1.62 g (65%) as a yellow oil. [α]=−37° (c 1%, MeOH); HRMS EI m/e 211.9840 (M)+, Calc'd. 211.9837

### Example 22

### (R)-2-Bromomethyl-7-(2,6-dichloro-phenyl)-5-fluoro-2,3-dihydro-benzofuran

Starting from 3-(3-bromo-2-hydroxy-propyl)-2′,6′,-dichloro-5-fluoro-biphenyl-2-ol (1.48 g, 3.7 mmol) and following the procedure described for Intermediate 19 gave the desired product 1.16 g (82%) as a clear oil. HRMS EI m/e 373.9277 (M)+, Calc'd. 373.9277; [α]−15.75° (c, 5.6 mg/0.7 ml, MeOH)

### Example 23

### (R)-2-Bromomethyl-7-(2-chloro-6-fluoro-phenyl)-5-fluoro-2,3-dihydro-benzofuran

Starting from (S)-3-(3-bromo-2-hydroxy-propyl)-2′-chloro-5,6′-difluorobiphenyl-2-ol (2.2 g, 5.8 mmol) and following the procedure described for Example 19 gave the desired product 2.12 g (100%) as a clear oil. MS APPI m/e 358 (M)⁺.

### Example 24

### (R)-7-Bromo-2-bromomethyl-5-fluoro-2,3-dihydrobenzofuran

To a solution of 2-bromomethyl-5-fluoro-2,3-dihydro-benzofuran (3.20 g, 14 mmol) in acetic acid was added bromine (2.2 ml, 42 mmol) at room temperature. The mixture was stirred at room temperature for overnight. The solvent was removed under the vacuum and the residue was washed with Na₂SO₃ and extracted with methylene chloride. Chromatography with 5% ethyl acetate in hexanes afforded product 3.16 g (74%) as a light yellow oil. HRMS EI m/e 307.8846 (M)+, Calc'd. 307.8848. [α]=+24.8 (c 1%, MeOH)

### Example 25

### (R)-2-Bromomethyl-5-fluoro-7-o-toly-2,3-dihydrobenzofuran

To a solution of 7-bromo-2-bromomethyl-5-fluoro-2,3-dihydrobenzofuran (2.57 g, 8.2 mmol) and o-tolyboronic acid (3.4 g, 24 mmol) in dioxane-water (4/1) was added dichlorobis(tri-o-tolyphosphine)-palladium (0.33 g, 0.41 mmol) and potassium carbonate (2.86 g, 21 mmol) at 90° C. The mixture was heated at 90° C. for 3 hours. The mixture was filtered through the pad of celite and concentrated under vacuum. Chromatography with 10-30% ethyl acetate in hexanes afforded product 2.54 g (95%) as a clear oil. HRMS EI m/e 320.0224 (M)+; [α]=+35.00° (c 1%, MeOH)

### Example 26

### (R)-2-Bromomethyl-7-(2-chloro-phenyl)-5-fluoro-2,3-dihydrobenzofuran

Starting from 7-bromo-2-bromomethyl-5-fluoro-2,3-dihydrobenzofuran (0.5 g, 1.6 mmol) and 2-chlorobenzene boronic acid (0.76 g, 4.8 mmol) and following the procedure described for Example 25 gave the desired product 0.55 g (99%) as a clear oil.

HRMS EI M+ 339.9657; [α]=+29.6° (c 5.7 mg/0.7 ml, MeOH)

### Example 27

### (R)-2-Bromomethyl-7-(2-methyl-5-chloro-phenyl)-5-fluoro-2,3-dihydrobenzofuran

Starting from 7-bromo-2-bromomethyl-5-fluoro-2,3-dihydrobenzofuran (0.40 g, 1.3 mmol) and 5-chloro-o-toluene boronic acid (0.88 g, 5.2 mmol) and following the procedure described for Intermediate 25 gave the desired product 0.41 g (90%) as a clear oil. HRMS EI M+ 353.9829; [α]=+47.38° (c 6.5 mg/0.7 ml, MeOH).

### Example 28

### (R)-2-Bromomethyl-7-(2-methyl-4-chloro-phenyl)-5-fluoro-2,3-dihydrobenzofuran

Starting from 7-bromo-2-bromomethyl-5-fluoro-2,3-dihydrobenzofuran (0.42 g, 1.3 mmol) and 4-chloro-o-toluene boronic acid (0.88 g, 5.2 mmol) and following the procedure described for Example 25 gave the desired product 0.43 g (95%) as a clear oil.

HRMS EI M+ 353.9825, Calc'd. 353.9825; [α]=39.14° (c 4.9 mg/0.7 ml, MeOH)

### Example 29

### (R)-2-Azidomethyl-7-(4-chloro-2-methyl-phenyl)-5-fluoro-2,3-dihydrobenzofuran

To a solution of 2-bromomethyl-7-(2-methyl-4-chloro-phenyl)-5-fluoro-2,3-dihydrobenzofuran (0.4 g, 1.1 mmol) in DMF was added sodium azide (0.33 g, 6.6 mmol). The mixture was heated at 90° C. overnight. The reaction was quenched with water. The mixture was extracted with methylene chloride. The organic layer was washed with water and dried over sodium sulfate. The organic solvent was removed under vacuum. Chromatography with 10% ethyl acetate in hexanes afforded product 0.30 g (85%) as a clear oil. HRMS EI m/e 317.0719 (M)+, Calc'd. 317.0718; [α]=+16.76° (c 6.1 mg/0.7 ml, MeOH)

### Example 30

### (R)-2-Azidomethyl-7-(5-chloro-2-methyl-phenyl)-5-fluoro-2,3-dihydrobenzofuran

Starting from 2-bromomethyl-7-(2-methyl-5-chloro-phenyl)-5-fluoro-2,3-dihydrobenzofuran (0.41 g, 1.2 mmol) following the procedure described for Example 29 gave arise the desired product 0.31 g (85%) as a clear oil. HRMS EI m/e 317.0734 (M)+, Calc'd. 317.0733; [α]=+3.12° (c 5.4 mg/0.7 ml, MeOH).

### Example 31

### (R)-2-Azidomethyl-7-(2-chloro-6-fluoro-phenyl)-5-fluoro-2,3-dihydrobenzofuran

Starting from (R)-2-bromomethyl-7-(2-chloro-6-fluoro-phenyl)-5-fluoro-2,3-dihydro-benzofuran (2.2 g, 5.8 mmol) following the procedure described for Example 29 gave arise the desired product 1.42 g (75%) as a clear oil. MS EI m/e 321 (M)+; [α]=+40.0° (1% solution in MeOH).

### Example 32

### (R)-(5-Fluoro-7-o-toly-2,3-dihydro-benzofuran-2-ylmethyl)methyl-amine

To a solution of 2-bromomethyl-5-fluoro-7-o-toly-2,3-dihydrobenzofuran (2.54 g, 7.9 mmol) in DMSO was added methyl amine (2.0 M in THF, 79 mmol)). The mixture was stirred at 50° C. for 10 hours. The mixture was extracted with methylene chloride and organic layer was washed with water. The solvent was removed under vacuum. The oil was dissolved in ethyl acetate and made into its hydrochloric salt using excess ethereal hydrochloric acid to give a white solid: mp. 145-147° C. [α]=+16.42° (c 5.2 mg/0.7 ml, MeOH)

Elemental Analysis for: C₁₇H₁₈FNO.1HCl Theory: C, 66.34 H, 6.22 N, 4.55 Found: C, 66.22 H, 6.20 N, 4.38

### Example 33

### (R)-[7-(2-Chloro-phenyl)-(5-fluoro-2,3-dihydro-benzofuran-2-ylmethyl)methyl-amine

Starting from 2-bromomethyl-7-(2-chloro-phenyl)-5-fluoro-2,3-dihydrobenzofuran (0.55 g, 1.6 mmol) following the procedure described for Example 32 gave arise the desired product 0.36 g (77%) as a clear oil. The oil was dissolved in ethyl acetate and made into its hydrochloric salt using excess ethereal hydrochloric acid to give a white foam. [α]=+11.57° (c 5.2 mg/0.7 ml, MeOH)

Elemental Analysis for: C₁₆H₁₅ClFNO.1HCl.1H₂O Theory: C, 55.51 H, 5.24N, 4.05 Found: C, 56.86 H, 5.27 N, 3.91

### Example 34

### (R)-[7-(2,6-dichloro-phenyl)-5-Fluoro-2,3-dihydro-benzofuran-2-ylmethyl]ethyl-amine

Starting from 2-bromomethyl-7-(2,6-dichloro-phenyl)-5-fluoro-2,3-dihydrobenzofuran (0.42 g, 1.1 mmol) and ethyl amine (2.0 M in THF, 5.6 ml, 11 mmol) following the procedure described for Example 32 gave the desired product 0.28 g (74%) as a clear oil. The oil was dissolved in ethyl acetate and made into its hydrochloric salt using excess ethereal hydrochloric acid to give a white foam. MS ES [M+H]+340.1; [α]=−7.12° (c 5.5 mg/0.7 ml, MeOH)

Elemental Analysis for: C₁₇H₁₆C₁₂FNO.1HCl.1H₂O; Theory: C, 51.73 H, 4.85N, 3.55 Found: C, 51.85 H, 4.88 N, 3.50

### Example 35

### (R)-[7-(2,6-Dichloro-phenyl)-5-fluoro-2,3-dihydro-benzofuran-2-ylmethyl]-dimethyl-amine

Starting from 2-bromomethyl-7-(2,6-dichloro-phenyl)-5-fluoro-2,3-dihydrobenzofuran (0.41 g, 1.1 mmol) and N,N-dimethyl amine (2.0 M in THF, 5.4 ml, 11 mmol) following the procedure described for Example 32 gave the desired product 0.29 g (80%) as a clear oil. The oil was dissolved in ethyl acetate and made into its hydrochloric salt using excess ethereal hydrochloric acid to give a white solid: mp. 156-158° C; [α]=−21.04° (c 5.4 mg/0.7 ml)

Elemental Analysis for C₁₇H₁₆Cl₂FNO.1HCl: Theory: C, 54.21 H, 4.55 N, 3.72 Found: C, 53.98 H, 4.62 N, 3.56

### Example 36

### (R)-C-[7-(5-Chloro-2-methyl phenyl)-5-fluoro-2,3-dihydro-benzofuran-2-yl]-methylamine

To a solution of 2-azidomethyl-7-(5-chloro-2-methyl-phenyl)-5-fluoro-2,3-dihydro-benzofuran (0.40 g, 1.2 mmol) in tetrahydrofuran was added polymer-supported triphenylphosphine (˜3 mmol/g, 3.6 mmol) and water. The mixture was stirred at room temperature for 24 hours, and filtered through the pad of celite. The solvent was removed under vacuum to form a clear oil. The oil was dissolved in ethyl acetate and made into its hydrochloric salt using excess ethereal hydrochloric acid to give a white solid: mp. 148-150° C.; [α]=+1.45° (c 5.8 mg/0.7 ml, MeOH)

Elemental Analysis for: C₁₆H₁₅ClFNO.1HCl Theory: C, 58.55 H, 4.91 N, 4.27 Found: C, 58.55 H, 4.78 N, 3.88

### Example 37

### (R)-C-[7-(4-Chloro-2-methyl phenyl)-5-fluoro-2,3-dihydro-benzofuran-2-yl]-methylamine

Starting from 2-azidomethyl-7-(4-chloro-2-methyl-phenyl)-5-fluoro-2,3-dihydrobenzo-furan (0.40 g, 1.2 mmol) following the procedure described for Example 36 gave the desired product 0.29 g (80%) as a clear oil. The oil was dissolved in ethyl acetate and made into its hydrochloric salt using excess ethereal hydrochloric acid to give a white solid: mp. 183-185° C; [α]=+7.22° (c 6.4 mg/0.7 ml, MeOH)

Elemental Analysis for: C₁₆H₁₅ClFNO.1HCl Theory: C, 58.55 H, 4.91 N, 4.27 Found: C, 58.55 H, 4.87 N, 4.52

### Example 38

### (R)-C-[7-(2-Chloro-6-fluoro-phenyl)-5-fluoro-2,3-dihydro-benzofuran-2-yl]-methylamine

Starting from (R)-2-azidomethyl-7-(2-chloro-6-fluoro-phenyl)-5-fluoro-2,3-dihydrobenzofuran (1.42 g, 4.4 mmol) following the procedure described for Example 36 gave the desired product 1.10 g (90%) as a clear oil. The oil was dissolved in ethyl acetate and made into its hydrochloric salt using excess ethereal hydrochloric acid to give a white solid: mp. 197-200° C.

Elemental Analysis for: C₁₅H₁₂ClF₂NO.1HCl Theory: C, 54.24 H, 3.95 N, 4.22 Found: C, 54.08 H, 3.83 N, 3.78

### Example 39

### ((2R)-7-(4-Chloro-2-methylphenyl)-6-fluoro-2,3-dihydrobenzofuran-2-yl)methanamine:

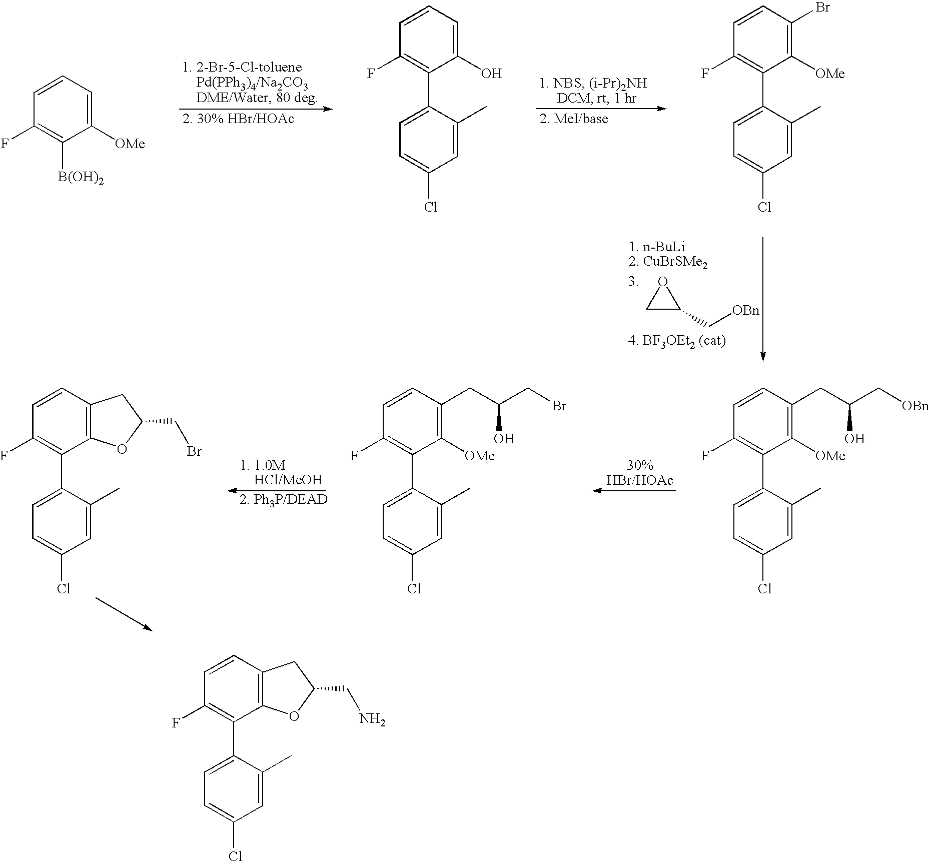

All patents, publications, and other documents cited herein are hereby incorporated by reference in their entirety.

We claim:

1. A method for preparing a compound of formula E: 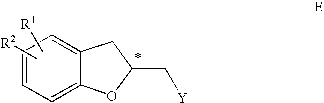
wherein:
R¹ and R² are each independently hydrogen, chlorine, fluorine, CN, —OH, C₁₋₈ alkyl, C₁-₆ perfluoroalkyl, C₁-₆ alkoxy, C₁-₆ perfluoroalkoxy, 6-10 membered aryl, 6-10 membered aryloxy, 5-10 membered heteroaryl having 1 to 4 heteroatoms independently selected from nitrogen, oxygen or sulfur, C₂-₈ alkenyl, C₁-₆ alkanesulfonamido, dialkylamino of 1 to 6 carbon atoms per alkyl moiety, C₃-₈ cycloaliphatic, or 3-8 membered heterocycloalkyl having 1 to 3 heteroatoms independently selected from nitrogen, oxygen or sulfur; or R¹ and R² when adjacent to each other may be taken together with the carbon atoms to which they are attached to form a cyclic moiety selected from a monocyclic cycloaliphatic of 3 to 8 carbon atoms, a bridged cycloaliphatic of 5 to 10 carbon atoms, a 3 to 8 membered heterocycloaliphatic having 1 to 3 heteroatoms each independently selected from nitrogen, oxygen, or sulfur, 6-10 membered aryl, or a 5-10 membered heteroaryl having 1 to 3 heteroatoms each independently selected from nitrogen, oxygen, or sulfur, wherein the monocyclic cycloaliphatic or the heterocycloaliphatic may be optionally substituted at a single carbon atom with a 3-5 membered cycloalkyl ring or a 3-5 membered heterocycloalkyl ring having 1-2 heteroatoms independently selected from nitrogen, oxygen, or sulfur, to form a spirocyclic group;
and
Y is Br, Cl, or I,
comprising the steps of:
(a) providing a chiral non-racemic compound of formula D: 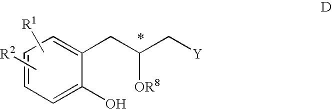
wherein:
Y is Br, Cl, or I; and
R⁸ is hydrogen or a suitable hydroxyl protecting group,
and
(b) cyclizing said compound of formula D to form a compound of formula E.

2. The method of claim 1 wherein the cyclizing step is accomplished using Mitsunobu reaction conditions.

3. The method of claim 1 further comprising the step of converting the compound of formula E to a compound of formula F: 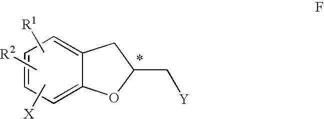
wherein:
R^{1 and R}² are each independently hydrogen, chlorine, fluorine, CN, —OH, C₁₋₈ alkyl, C₁-₆ perfluoroalkyl, C₁-₆ alkoxy, C₁-₆ perfluoroalkoxy, 6-10 membered aryl, 6-10 membered aryloxy, 5-10 membered heteroaryl having 1 to 4 heteroatoms independently selected from nitrogen, oxygen or sulfur, C₂-₈ alkenyl, C₁-₆ alkanesulfonamido, dialkylamino of 1 to 6 carbon atoms per alkyl moiety, C₃-₈ cycloaliphatic, or 3-8 membered heterocycloalkyl having 1 to 3 heteroatoms independently selected from nitrogen, oxygen or sulfur; or R¹ and R² when adjacent to each other may be taken together with the carbon atoms to which they are attached to form a cyclic moiety selected from a monocyclic cycloaliphatic of 3 to 8 carbon atoms, a bridged cycloaliphatic of 5 to 10 carbon atoms, a 3 to 8 membered heterocycloaliphatic having 1 to 3 heteroatoms each independently selected from nitrogen, oxygen, or sulfur, 6-10 membered aryl, or a 5-10 membered heteroaryl having 1 to 3 heteroatoms each independently selected from nitrogen, oxygen, or sulfur, wherein the monocyclic cycloaliphatic or the heterocycloaliphatic may be optionally substituted at a single carbon atom with a 3-5 membered cycloalkyl ring or a 3-5 membered heterocycloalkyl ring having 1-2 heteroatoms independently selected from nitrogen, oxygen, or sulfur, to form a spirocyclic group;
Y is Br, Cl, or I; and
X is halogen or triflate.

4. The method of claim 3 wherein the step of converting the compound of formula E to a compound of formula F comprises the steps of:
(a) formylating the compound of formula E to provide a formyl group,
(b) converting the formyl group to a hydroxyl group via Baeyer-Villiger conditions, and
(c) triflating the resulting hydroxyl group.

5. The method of claim 1 wherein the compound of formula D is prepared by providing a compound of formula C′: 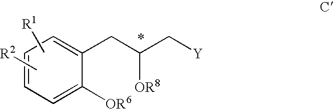
wherein:
R¹ and R² are each independently hydrogen, chlorine, fluorine, CN, —OH, C₁₋₈ alkyl, C₁-₆ perfluoroalkyl, C₁-₆ alkoxy, C₁-₆ perfluoroalkoxy, 6-10 membered aryl, 6-10 membered aryloxy, 5-10 membered heteroaryl having 1 to 4 heteroatoms independently selected from nitrogen, oxygen or sulfur, C₂-₈ alkenyl, C₁-₆ alkanesulfonamido, dialkylamino of 1 to 6 carbon atoms per alkyl moiety, C₃-₈ cycloaliphatic, or 3-8 membered heterocycloalkyl having 1 to 3 heteroatoms independently selected from nitrogen, oxygen or sulfur; or R¹ and R² when adjacent to each other may be taken together with the carbon atoms to which they are attached to form a cyclic moiety selected from a monocyclic cycloaliphatic of 3 to 8 carbon atoms, a bridged cycloaliphatic of 5 to 10 carbon atoms, a 3 to 8 membered heterocycloaliphatic having 1 to 3 heteroatoms each independently selected from nitrogen, oxygen, or sulfur, 6-10 membered aryl, or a 5-10 membered heteroaryl having 1 to 3 heteroatoms each independently selected from nitrogen, oxygen, or sulfur, wherein the monocyclic cycloaliphatic or the heterocycloaliphatic may be optionally substituted at a single carbon atom with a 3-5 membered cycloalkyl ring or a 3-5 membered heterocycloalkyl ring having 1-2 heteroatoms independently selected from nitrogen, oxygen, or sulfur, to form a spirocyclic group;
Y is Br, Cl, or I;
R⁶ is a suitable hydroxyl protecting group; and
R⁸ is hydrogen or a suitable hydroxyl protecting group,
and removing the R⁶ protecting group from the compound of formula C′ to produce a compound of formula D.

6. The method of claim 3 further comprising the step of converting the compound of formula F to a compound of formula I: 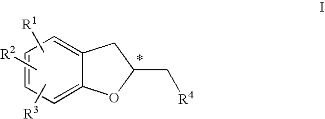
or a pharmaceutically acceptable salt thereof, wherein:
R¹ and R² are each independently hydrogen, chlorine, fluorine, CN, —OH, C₁₋₈ alkyl, C₁-₆ perfluoroalkyl, C₁-₆ alkoxy, C₁-₆ perfluoroalkoxy, 6-10 membered aryl, 6-10 membered aryloxy, 5-10 membered heteroaryl having 1 to 4 heteroatoms independently selected from nitrogen, oxygen or sulfur, C₂-₈ alkenyl, C₁-₆ alkanesulfonamido, dialkylamino of 1 to 6 carbon atoms per alkyl moiety, C₃-₈ cycloaliphatic, or 3-8 membered heterocycloalkyl having 1 to 3 heteroatoms independently selected from nitrogen, oxygen or sulfur; or R¹ and R² when adjacent to each other may be taken together with the carbon atoms to which they are attached to form a cyclic moiety selected from a monocyclic cycloaliphatic of 3 to 8 carbon atoms, a bridged cycloaliphatic of 5 to 10 carbon atoms, a 3 to 8 membered heterocycloaliphatic having 1 to 3 heteroatoms each independently selected from nitrogen, oxygen, or sulfur, 6-10 membered aryl, or a 5-10 membered heteroaryl having 1 to 3 heteroatoms each independently selected from nitrogen, oxygen, or sulfur, wherein the monocyclic cycloaliphatic or the heterocycloaliphatic may be optionally substituted at a single carbon atom with a 3-5 membered cycloalkyl ring or a 3-5 membered heterocycloalkyl ring having 1-2 heteroatoms independently selected from nitrogen, oxygen, or sulfur, to form a spirocyclic group;
R³ is hydrogen, 6-10 membered aryl, or 5-10 membered heteroaryl having 1 to 4 heteroatoms independently selected from nitrogen, oxygen or sulfur, wherein R³ is optionally substituted with one or more R^{x} groups;
each R^{x} is independently selected from halogen, OH, lower alkyl, lower alkoxy, lower haloalkyl, lower haloalkoxy, or CN;
R⁴ is CN, N₃, or N(R⁵)(R^{5a}); and
R⁵ and R^{5a} are each independently hydrogen, an amine protecting group, C₁₋₆ alkyl, lower haloalkyl, 3-6 membered cycloaliphatic, or alkylcycloaliphatic, or R⁵ and R^{5a} are taken together with the nitrogen to which they are attached to form a cyclic amine protecting group or a 3-6 membered saturated or partially unsaturated ring having 1-2 heteroatoms independently selected from nitrogen, oxygen, or sulfur.

7. The method of claim 1 wherein conversion of the compound of formula D to the compound of formula E comprises the steps of:
(a) removing the R⁸ hydroxyl protecting group from the compound of formula D to produce a compound of the formula D-1: 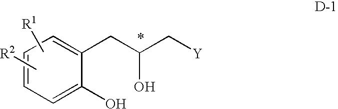
wherein:
R¹ and R² are each independently hydrogen, chlorine, fluorine, CN, —OH, C₁₋₈ alkyl, C₁-₆ perfluoroalkyl, C₁-₆ alkoxy, C₁-₆ perfluoroalkoxy, 6-10 membered aryl, 6-10 membered aryloxy, 5-10 membered heteroaryl having 1 to 4 heteroatoms independently selected from nitrogen, oxygen or sulfur, C₂-₈ alkenyl, C₁-₆ alkanesulfonamido, dialkylamino of 1 to 6 carbon atoms per alkyl moiety, C₃-₈ cycloaliphatic, or 3-8 membered heterocycloalkyl having 1 to 3 heteroatoms independently selected from nitrogen, oxygen or sulfur; or R¹ and R² when adjacent to each other may be taken together with the carbon atoms to which they are attached to form a cyclic moiety selected from a monocyclic cycloaliphatic of 3 to 8 carbon atoms, a bridged cycloaliphatic of 5 to 10 carbon atoms, a 3 to 8 membered heterocycloaliphatic having 1 to 3 heteroatoms each independently selected from nitrogen, oxygen, or sulfur, 6-10 membered aryl, or a 5-10 membered heteroaryl having 1 to 3 heteroatoms each independently selected from nitrogen, oxygen, or sulfur, wherein the monocyclic cycloaliphatic or the heterocycloaliphatic may be optionally substituted at a single carbon atom with a 3-5 membered cycloalkyl ring or a 3-5 membered heterocycloalkyl ring having 1-2 heteroatoms independently selected from nitrogen, oxygen, or sulfur, to form a spirocyclic group; and
Y is Br, Cl, or I;
and
(b) cyclizing the compound of formula D-1 to produce a compound of formula E: 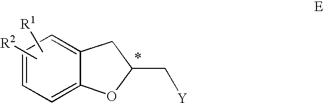
wherein:
R¹ and R² are each independently hydrogen, chlorine, fluorine, CN, —OH, C₁₋₈ alkyl, C₁-₆ perfluoroalkyl, C₁-₆ alkoxy, C₁-₆ perfluoroalkoxy, 6-10 membered aryl, 6-10 membered aryloxy, 5-10 membered heteroaryl having 1 to 4 heteroatoms independently selected from nitrogen, oxygen or sulfur, C₂-₈ alkenyl, C₁-₆ alkanesulfonamido, dialkylamino of 1 to 6 carbon atoms per alkyl moiety, C₃-₈ cycloaliphatic, or 3-8 membered heterocycloalkyl having 1 to 3 heteroatoms independently selected from nitrogen, oxygen or sulfur; or R¹ and R² when adjacent to each other may be taken together with the carbon atoms to which they are attached to form a cyclic moiety selected from a monocyclic cycloaliphatic of 3 to 8 carbon atoms, a bridged cycloaliphatic of 5 to 10 carbon atoms, a 3 to 8 membered heterocycloaliphatic having 1 to 3 heteroatoms each independently selected from nitrogen, oxygen, or sulfur, 6-10 membered aryl, or a 5-10 membered heteroaryl having 1 to 3 heteroatoms each independently selected from nitrogen, oxygen, or sulfur, wherein the monocyclic cycloaliphatic or the heterocycloaliphatic may be optionally substituted at a single carbon atom with a 3-5 membered cycloalkyl ring or a 3-5 membered heterocycloalkyl ring having 1-2 heteroatoms independently selected from nitrogen, oxygen, or sulfur, to form a spirocyclic group; and
Y is Br, Cl, or I.

8. The method of claim 6 wherein conversion of the compound of formula F to the compound of formula I comprises the steps of:
(a) converting the compound of formula F to a compound of formula G: 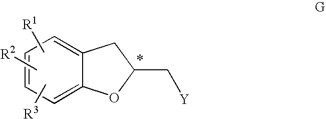
wherein:
R¹ and R² are each independently hydrogen, chlorine, fluorine, CN, —OH, C₁₋₈ alkyl, C₁-₆ perfluoroalkyl, C₁-₆ alkoxy, C₁-₆ perfluoroalkoxy, 6-10 membered aryl, 6-10 membered aryloxy, 5-10 membered heteroaryl having 1 to 4 heteroatoms independently selected from nitrogen, oxygen or sulfur, C₂-₈ alkenyl, C₁-₆ alkanesulfonamido, dialkylamino of 1 to 6 carbon atoms per alkyl moiety, C₃-₈ cycloaliphatic, or 3-8 membered heterocycloalkyl having 1 to 3 heteroatoms independently selected from nitrogen, oxygen or sulfur; or R¹ and R² when adjacent to each other may be taken together with the carbon atoms to which they are attached to form a cyclic moiety selected from a monocyclic cycloaliphatic of 3 to 8 carbon atoms, a bridged cycloaliphatic of 5 to 10 carbon atoms, a 3 to 8 membered heterocycloaliphatic having 1 to 3 heteroatoms each independently selected from nitrogen, oxygen, or sulfur, 6-10 membered aryl, or a 5-10 membered heteroaryl having 1 to 3 heteroatoms each independently selected from nitrogen, oxygen, or sulfur, wherein the monocyclic cycloaliphatic or the heterocycloaliphatic may be optionally substituted at a single carbon atom with a 3-5 membered cycloalkyl ring or a 3-5 membered heterocycloalkyl ring having 1-2 heteroatoms independently selected from nitrogen, oxygen, or sulfur, to form a spirocyclic group;
R³ is hydrogen, 6-10 membered aryl, or 5-10 membered heteroaryl having 1 to 4 heteroatoms independently selected from nitrogen, oxygen or sulfur, wherein R³ is optionally substituted with one or more R^{x} groups;
each R^{x} is independently selected from halogen, OH, lower alkyl, lower alkoxy, lower haloalkyl, lower haloalkoxy, or CN; and
Y is Br, Cl, or I;
and
(b) reacting the compound of formula G with an amine or a protected amine to produce a compound of formula I.

9. The method of claim 8 wherein step (a) is achieved via a Suzuki reaction.

10. The method of claim 6 wherein conversion of the compound of formula F to a compound of formula I comprises the steps of:
(a) converting the compound of formula F to a compound of formula G: 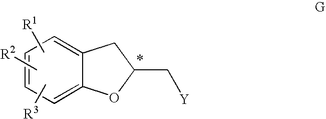
wherein:
R¹ and R² are each independently hydrogen, chlorine, fluorine, CN, —OH, C₁₋₈ alkyl, C₁-₆ perfluoroalkyl, C₁-₆ alkoxy, C₁-₆ perfluoroalkoxy, 6-10 membered aryl, 6-10 membered aryloxy, 5-10 membered heteroaryl having 1 to 4 heteroatoms independently selected from nitrogen, oxygen or sulfur, C₂-₈ alkenyl, C₁-₆ alkanesulfonamido, dialkylamino of 1 to 6 carbon atoms per alkyl moiety, C₃-₈ cycloaliphatic, or 3-8 membered heterocycloalkyl having 1 to 3 heteroatoms independently selected from nitrogen, oxygen or sulfur; or R¹ and R² when adjacent to each other may be taken together with the carbon atoms to which they are attached to form a cyclic moiety selected from a monocyclic cycloaliphatic of 3 to 8 carbon atoms, a bridged cycloaliphatic of 5 to 10 carbon atoms, a 3 to 8 membered heterocycloaliphatic having 1 to 3 heteroatoms each independently selected from nitrogen, oxygen, or sulfur, 6-10 membered aryl, or a 5-10 membered heteroaryl having 1 to 3 heteroatoms each independently selected from nitrogen, oxygen, or sulfur, wherein the monocyclic cycloaliphatic or the heterocycloaliphatic may be optionally substituted at a single carbon atom with a 3-5 membered cycloalkyl ring or a 3-5 membered heterocycloalkyl ring having 1-2 heteroatoms independently selected from nitrogen, oxygen, or sulfur, to form a spirocyclic group;
R³ is hydrogen, 6-10 membered aryl, or 5-10 membered heteroaryl having 1 to 4 heteroatoms independently selected from nitrogen, oxygen or sulfur, wherein R³ is optionally substituted with one or more R^{x} groups;
each R^{x} is independently selected from halogen, OH, lower alkyl, lower alkoxy, lower haloalkyl, lower haloalkoxy, or CN; and
Y is Br, Cl, or I,
(b) reacting the compound of formula G with an alkali metal azide to produce a compound of formula G-1: 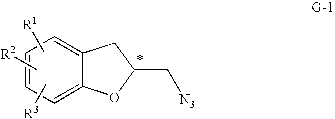
wherein:
R¹ and R² are each independently hydrogen, chlorine, fluorine, CN, —OH, C₁₋₈ alkyl, C₁-₆ perfluoroalkyl, C₁-₆ alkoxy, C₁-₆ perfluoroalkoxy, 6-10 membered aryl, 6-10 membered aryloxy, 5-10 membered heteroaryl having 1 to 4 heteroatoms independently selected from nitrogen, oxygen or sulfur, C₂-₈ alkenyl, C₁-₆ alkanesulfonamido, dialkylamino of 1 to 6 carbon atoms per alkyl moiety, C₃-₈ cycloaliphatic, or 3-8 membered heterocycloalkyl having 1 to 3 heteroatoms independently selected from nitrogen, oxygen or sulfur; or R¹ and R² when adjacent to each other may be taken together with the carbon atoms to which they are attached to form a cyclic moiety selected from a monocyclic cycloaliphatic of 3 to 8 carbon atoms, a bridged cycloaliphatic of 5 to 10 carbon atoms, a 3 to 8 membered heterocycloaliphatic having 1 to 3 heteroatoms each independently selected from nitrogen, oxygen, or sulfur, 6-10 membered aryl, or a 5-10 membered heteroaryl having 1 to 3 heteroatoms each independently selected from nitrogen, oxygen, or sulfur, wherein the monocyclic cycloaliphatic or the heterocycloaliphatic may be optionally substituted at a single carbon atom with a 3-5 membered cycloalkyl ring or a 3-5 membered heterocycloalkyl ring having 1-2 heteroatoms independently selected from nitrogen, oxygen, or sulfur, to form a spirocyclic group; and
R³ is hydrogen, 6-10 membered aryl, or 5-10 membered heteroaryl having 1 to 4 heteroatoms independently selected from nitrogen, oxygen or sulfur, wherein R³ is optionally substituted with one or more R^{x} groups;
each R^{x} is independently selected from halogen, OH, lower alkyl, lower alkoxy, lower haloalkyl, lower haloalkoxy, or CN;
and
(c) reducing the compound of formula G-1 to produce a compound of formula I where R⁴ is NH₂.

11. The method of claim 1 wherein the step of providing the compound of formula D comprises the steps of:
(a) providing a compound of formula A: 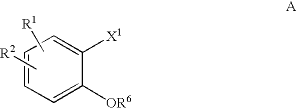
wherein:
R¹ and R² are each independently hydrogen, chlorine, fluorine, CN, —OH, C₁₋₈ alkyl, C₁-₆ perfluoroalkyl, C₁-₆ alkoxy, C₁-₆ perfluoroalkoxy, 6-10 membered aryl, 6-10 membered aryloxy, 5-10 membered heteroaryl having 1 to 4 heteroatoms independently selected from nitrogen, oxygen or sulfur, C₂-₈ alkenyl, C₁-₆ alkanesulfonamido, dialkylamino of 1 to 6 carbon atoms per alkyl moiety, C₃-₈ cycloaliphatic, or 3-8 membered heterocycloalkyl having 1 to 3 heteroatoms independently selected from nitrogen, oxygen or sulfur; or R¹ and R² when adjacent to each other may be taken together with the carbon atoms to which they are attached to form a cyclic moiety selected from a monocyclic cycloaliphatic of 3 to 8 carbon atoms, a bridged cycloaliphatic of 5 to 10 carbon atoms, a 3 to 8 membered heterocycloaliphatic having 1 to 3 heteroatoms each independently selected from nitrogen, oxygen, or sulfur, 6-10 membered aryl, or a 5-10 membered heteroaryl having 1 to 3 heteroatoms each independently selected from nitrogen, oxygen, or sulfur, wherein the monocyclic cycloaliphatic or the heterocycloaliphatic may be optionally substituted at a single carbon atom with a 3-5 membered cycloalkyl ring or a 3-5 membered heterocycloalkyl ring having 1-2 heteroatoms independently selected from nitrogen, oxygen, or sulfur, to form a spirocyclic group;
R⁶ is a suitable hydroxyl protecting group; and
X¹ is halogen,
(b) treating the compound of formula A with an chiral non-racemic compound of formula B: 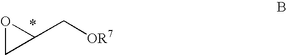
wherein R is a suitable hydroxyl protecting group;
to form a compound of formula C: 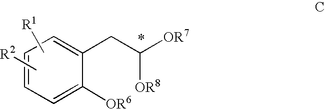
R¹ and R² are each independently hydrogen, chlorine, fluorine, CN, —OH, C₁₋₈ alkyl, C₁-₆ perfluoroalkyl, C₁-₆ alkoxy, C₁-₆ perfluoroalkoxy, 6-10 membered aryl, 6-10 membered aryloxy, 5-10 membered heteroaryl having 1 to 4 heteroatoms independently selected from nitrogen, oxygen or sulfur, C₂-₈ alkenyl, C₁-₆ alkanesulfonamido, dialkylamino of 1 to 6 carbon atoms per alkyl moiety, C₃-₈ cycloaliphatic, or 3-8 membered heterocycloalkyl having 1 to 3 heteroatoms independently selected from nitrogen, oxygen or sulfur; or R¹ and R² when adjacent to each other may be taken together with the carbon atoms to which they are attached to form a cyclic moiety selected from a monocyclic cycloaliphatic of 3 to 8 carbon atoms, a bridged cycloaliphatic of 5 to 10 carbon atoms, a 3 to 8 membered heterocycloaliphatic having 1 to 3 heteroatoms each independently selected from nitrogen, oxygen, or sulfur, 6-10 membered aryl, or a 5-10 membered heteroaryl having 1 to 3 heteroatoms each independently selected from nitrogen, oxygen, or sulfur, wherein the monocyclic cycloaliphatic or the heterocycloaliphatic may be optionally substituted at a single carbon atom with a 3-5 membered cycloalkyl ring or a 3-5 membered heterocycloalkyl ring having 1-2 heteroatoms independently selected from nitrogen, oxygen, or sulfur, to form a spirocyclic group;
R⁶ is a suitable hydroxyl protecting group;
R⁷ is an acid-labile hydroxyl protecting group; and
R⁸ is hydrogen or a hydroxyl protecting group,
and
(c) reacting the compound of formula C with a hydrogen halide to produce a compound of formula D.

12. The method of claim 11 wherein the conversion of the compound of formula A to the compound of formula D comprises the steps of:
(a) treating a compound of the formula A with an chiral non-racemic compound of formula B: 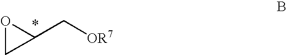
wherein R is a suitable hydroxyl protecting group;
to form a compound of formula C-1: 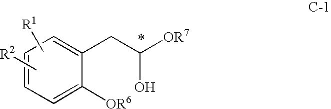
wherein:
R¹ and R² are each independently hydrogen, chlorine, fluorine, CN, —OH, C₁₋₈ alkyl, C₁-₆ perfluoroalkyl, C₁-₆ alkoxy, C₁-₆ perfluoroalkoxy, 6-10 membered aryl, 6-10 membered aryloxy, 5-10 membered heteroaryl having 1 to 4 heteroatoms independently selected from nitrogen, oxygen or sulfur, C₂-₈ alkenyl, C₁-₆ alkanesulfonamido, dialkylamino of 1 to 6 carbon atoms per alkyl moiety, C₃-₈ cycloaliphatic, or 3-8 membered heterocycloalkyl having 1 to 3 heteroatoms independently selected from nitrogen, oxygen or sulfur; or R¹ and R² when adjacent to each other may be taken together with the carbon atoms to which they are attached to form a cyclic moiety selected from a monocyclic cycloaliphatic of 3 to 8 carbon atoms, a bridged cycloaliphatic of 5 to 10 carbon atoms, a 3 to 8 membered heterocycloaliphatic having 1 to 3 heteroatoms each independently selected from nitrogen, oxygen, or sulfur, 6-10 membered aryl, or a 5-10 membered heteroaryl having 1 to 3 heteroatoms each independently selected from nitrogen, oxygen, or sulfur, wherein the monocyclic cycloaliphatic or the heterocycloaliphatic may be optionally substituted at a single carbon atom with a 3-5 membered cycloalkyl ring or a 3-5 membered heterocycloalkyl ring having 1-2 heteroatoms independently selected from nitrogen, oxygen, or sulfur, to form a spirocyclic group;
R⁶ is a suitable hydroxyl protecting group; and
R⁷ is a hydroxyl protecting group;
and
(b) converting the compound C-1 to a compound of formula D.

13. The method of claim 12 wherein conversion of compound A to compound C-1 comprises performing a metal-halogen exchange reaction followed by forming an organocuprate.

14. The method of claim 13 wherein the metal-halogen exchange reaction utilizes at least one of n-butyl lithium or iso-propyl magnesium chloride.

15. The method of claim 14 wherein the organocuprate is formed utilizing CuBrSMe₂ or CuCN.

16. A method for preparing a compound of formula I: 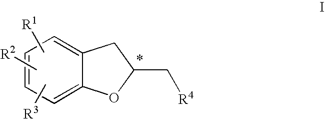
or a pharmaceutically acceptable salt thereof, wherein:
R¹ and R² are each independently hydrogen, chlorine, fluorine, CN, —OH, C₁₋₈ alkyl, C₁-₆ perfluoroalkyl, C₁-₆ alkoxy, C₁-₆ perfluoroalkoxy, 6-10 membered aryl, 6-10 membered aryloxy, 5-10 membered heteroaryl having 1 to 4 heteroatoms independently selected from nitrogen, oxygen or sulfur, C₂-₈ alkenyl, C₁-₆ alkanesulfonamido, dialkylamino of 1 to 6 carbon atoms per alkyl moiety, C₃-₈ cycloaliphatic, or 3-8 membered heterocycloalkyl having 1 to 3 heteroatoms independently selected from nitrogen, oxygen or sulfur; or R¹ and R² when adjacent to each other may be taken together with the carbon atoms to which they are attached to form a cyclic moiety selected from a monocyclic cycloaliphatic of 3 to 8 carbon atoms, a bridged cycloaliphatic of 5 to 10 carbon atoms, a 3 to 8 membered heterocycloaliphatic having 1 to 3 heteroatoms each independently selected from nitrogen, oxygen, or sulfur, 6-10 membered aryl, or a 5-10 membered heteroaryl having 1 to 3 heteroatoms each independently selected from nitrogen, oxygen, or sulfur, wherein the monocyclic cycloaliphatic or the heterocycloaliphatic may be optionally substituted at a single carbon atom with a 3-5 membered cycloalkyl ring or a 3-5 membered heterocycloalkyl ring having 1-2 heteroatoms independently selected from nitrogen, oxygen, or sulfur, to form a spirocyclic group;
R³ is hydrogen, 6-10 membered aryl, or 5-10 membered heteroaryl having 1 to 4 heteroatoms independently selected from nitrogen, oxygen or sulfur, wherein R³ is optionally substituted with one or more R^{x} groups;
each R^{x} is independently selected from halogen, OH, lower alkyl, lower alkoxy, lower haloalkyl, lower haloalkoxy, or CN;
R⁴ is CN, N₃, or N(R⁵)(R^{5a}); and
R⁵ and R^{5a} are each independently hydrogen, an amine protecting group, C₁₋₆ alkyl, lower haloalkyl, 3-6 membered cycloaliphatic, or alkylcycloaliphatic, or R⁵ and R^{5a} are taken together with the nitrogen to which they are attached to form a cyclic amine protecting group or a 3-6 membered saturated or partially unsaturated ring having 1-2 heteroatoms independently selected from nitrogen, oxygen, or sulfur.
comprising the steps of:
(a) providing a compound of formula D: 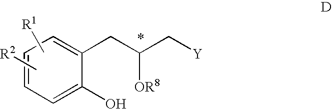
wherein:
R¹ and R² are each independently hydrogen, chlorine, fluorine, CN, —OH, C₁₋₈ alkyl, C₁-₆ perfluoroalkyl, C₁-₆ alkoxy, C₁-₆ perfluoroalkoxy, 6-10 membered aryl, 6-10 membered aryloxy, 5-10 membered heteroaryl having 1 to 4 heteroatoms independently selected from nitrogen, oxygen or sulfur, C₂-₈ alkenyl, C₁-₆ alkanesulfonamido, dialkylamino of 1 to 6 carbon atoms per alkyl moiety, C₃-₈ cycloaliphatic, or 3-8 membered heterocycloalkyl having 1 to 3 heteroatoms independently selected from nitrogen, oxygen or sulfur; or R¹ and R² when adjacent to each other may be taken together with the carbon atoms to which they are attached to form a cyclic moiety selected from a monocyclic cycloaliphatic of 3 to 8 carbon atoms, a bridged cycloaliphatic of 5 to 10 carbon atoms, a 3 to 8 membered heterocycloaliphatic having 1 to 3 heteroatoms each independently selected from nitrogen, oxygen, or sulfur, 6-10 membered aryl, or a 5-10 membered heteroaryl having 1 to 3 heteroatoms each independently selected from nitrogen, oxygen, or sulfur, wherein the monocyclic cycloaliphatic or the heterocycloaliphatic may be optionally substituted at a single carbon atom with a 3-5 membered cycloalkyl ring or a 3-5 membered heterocycloalkyl ring having 1-2 heteroatoms independently selected from nitrogen, oxygen, or sulfur, to form a spirocyclic group;
Y is Br, Cl, or I; and
R⁸ is hydrogen or a base-labile hydroxyl protecting group,
(b) converting the compound of formula D to a compound of the formula F-1: 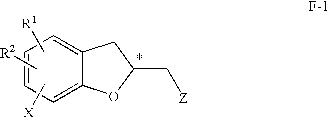
R¹ and R² are each independently hydrogen, chlorine, fluorine, CN, —OH, C₁₋₈ alkyl, C₁-₆ perfluoroalkyl, C₁-₆ alkoxy, C₁-₆ perfluoroalkoxy, 6-10 membered aryl, 6-10 membered aryloxy, 5-10 membered heteroaryl having 1 to 4 heteroatoms independently selected from nitrogen, oxygen or sulfur, C₂-₈ alkenyl, C₁-₆ alkanesulfonamido, dialkylamino of 1 to 6 carbon atoms per alkyl moiety, C₃-₈ cycloaliphatic, or 3-8 membered heterocycloalkyl having 1 to 3 heteroatoms independently selected from nitrogen, oxygen or sulfur; or R¹ and R² when adjacent to each other may be taken together with the carbon atoms to which they are attached to form a cyclic moiety selected from a monocyclic cycloaliphatic of 3 to 8 carbon atoms, a bridged cycloaliphatic of 5 to 10 carbon atoms, a 3 to 8 membered heterocycloaliphatic having 1 to 3 heteroatoms each independently selected from nitrogen, oxygen, or sulfur, 6-10 membered aryl, or a 5-10 membered heteroaryl having 1 to 3 heteroatoms each independently selected from nitrogen, oxygen, or sulfur, wherein the monocyclic cycloaliphatic or the heterocycloaliphatic may be optionally substituted at a single carbon atom with a 3-5 membered cycloalkyl ring or a 3-5 membered heterocycloalkyl ring having 1-2 heteroatoms independently selected from nitrogen, oxygen, or sulfur, to form a spirocyclic group;
X is halogen or triflate; and
Z is a suitable leaving group,
and
(c) converting the compound of formula F-1 to a compound of formula 1.

17. The method of claim 16 wherein conversion of the compound of formula D to the compound of formula F-1 comprises the steps of:
(a) cyclizing the compound of formula D by reacting with base to produce a compound of formula E-1: 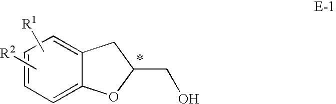
wherein:
R¹ and R² are each independently hydrogen, chlorine, fluorine, CN, —OH, C₁₋₈ alkyl, C₁-₆ perfluoroalkyl, C₁-₆ alkoxy, C₁-₆ perfluoroalkoxy, 6-10 membered aryl, 6-10 membered aryloxy, 5-10 membered heteroaryl having 1 to 4 heteroatoms independently selected from nitrogen, oxygen or sulfur, C₂-₈ alkenyl, C₁-₆ alkanesulfonamido, dialkylamino of 1 to 6 carbon atoms per alkyl moiety, C₃-₈ cycloaliphatic, or 3-8 membered heterocycloalkyl having 1 to 3 heteroatoms independently selected from nitrogen, oxygen or sulfur; or R¹ and R² when adjacent to each other may be taken together with the carbon atoms to which they are attached to form a cyclic moiety selected from a monocyclic cycloaliphatic of 3 to 8 carbon atoms, a bridged cycloaliphatic of 5 to 10 carbon atoms, a 3 to 8 membered heterocycloaliphatic having 1 to 3 heteroatoms each independently selected from nitrogen, oxygen, or sulfur, 6-10 membered aryl, or a 5-10 membered heteroaryl having 1 to 3 heteroatoms each independently selected from nitrogen, oxygen, or sulfur, wherein the monocyclic cycloaliphatic or the heterocycloaliphatic may be optionally substituted at a single carbon atom with a 3-5 membered cycloalkyl ring or a 3-5 membered heterocycloalkyl ring having 1-2 heteroatoms independently selected from nitrogen, oxygen, or sulfur, to form a spirocyclic group;
(b) converting the hydroxyl group of the compound of formula E-1 to a leaving group to provide a compound of the formula E-2: 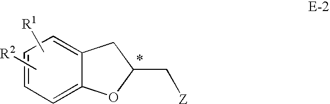
wherein:
R¹ and R² are each independently hydrogen, chlorine, fluorine, CN, —OH, C₁₋₈ alkyl, C₁-₆ perfluoroalkyl, C₁-₆ alkoxy, C₁-₆ perfluoroalkoxy, 6-10 membered aryl, 6-10 membered aryloxy, 5-10 membered heteroaryl having 1 to 4 heteroatoms independently selected from nitrogen, oxygen or sulfur, C₂-₈ alkenyl, C₁-₆ alkanesulfonamido, dialkylamino of 1 to 6 carbon atoms per alkyl moiety, C₃-₈ cycloaliphatic, or 3-8 membered heterocycloalkyl having 1 to 3 heteroatoms independently selected from nitrogen, oxygen or sulfur; or R¹ and R² when adjacent to each other may be taken together with the carbon atoms to which they are attached to form a cyclic moiety selected from a monocyclic cycloaliphatic of 3 to 8 carbon atoms, a bridged cycloaliphatic of 5 to 10 carbon atoms, a 3 to 8 membered heterocycloaliphatic having 1 to 3 heteroatoms each independently selected from nitrogen, oxygen, or sulfur, 6-10 membered aryl, or a 5-10 membered heteroaryl having 1 to 3 heteroatoms each independently selected from nitrogen, oxygen, or sulfur, wherein the monocyclic cycloaliphatic or the heterocycloaliphatic may be optionally substituted at a single carbon atom with a 3-5 membered cycloalkyl ring or a 3-5 membered heterocycloalkyl ring having 1-2 heteroatoms independently selected from nitrogen, oxygen, or sulfur, to form a spirocyclic group; and
Z is a suitable leaving group;
and
(c) converting the compound of formula E-2 to a compound of formula F-1.

18. The method of claim 17 wherein conversion of the compound of formula E-2 to a compound of formula F-1 comprises either of the steps of:
(a) formylating the compound of formula E-2 to provide a formyl group, converting the formyl group to a hydroxyl group via a Baeyer-Villiger procedure, and triflating the resulting hydroxyl group with trifluoromethanesulfonic anhydride in the presence of a tertiary amine to form a compound of formula F-1 wherein X is triflate, or
(b) contacting a compound of formula E-2 with a halogenating agent to form a compound of formula F-1 wherein X is halogen.

19. The method of claim 16 wherein conversion of the compound of formula F-1 to the compound of formula I comprises the steps of:
(a) converting the compound of formula F-1 to a compound of formula G-1: 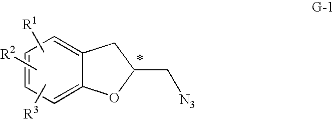
R¹ and R² are each independently hydrogen, chlorine, fluorine, CN, —OH, C₁₋₈ alkyl, C₁-₆ perfluoroalkyl, C₁-₆ alkoxy, C₁-₆ perfluoroalkoxy, 6-10 membered aryl, 6-10 membered aryloxy, 5-10 membered heteroaryl having 1 to 4 heteroatoms independently selected from nitrogen, oxygen or sulfur, C₂-₈ alkenyl, C₁-₆ alkanesulfonamido, dialkylamino of 1 to 6 carbon atoms per alkyl moiety, C₃-₈ cycloaliphatic, or 3-8 membered heterocycloalkyl having 1 to 3 heteroatoms independently selected from nitrogen, oxygen or sulfur; or R¹ and R² when adjacent to each other may be taken together with the carbon atoms to which they are attached to form a cyclic moiety selected from a monocyclic cycloaliphatic of 3 to 8 carbon atoms, a bridged cycloaliphatic of 5 to 10 carbon atoms, a 3 to 8 membered heterocycloaliphatic having 1 to 3 heteroatoms each independently selected from nitrogen, oxygen, or sulfur, 6-10 membered aryl, or a 5-10 membered heteroaryl having 1 to 3 heteroatoms each independently selected from nitrogen, oxygen, or sulfur, wherein the monocyclic cycloaliphatic or the heterocycloaliphatic may be optionally substituted at a single carbon atom with a 3-5 membered cycloalkyl ring or a 3-5 membered heterocycloalkyl ring having 1-2 heteroatoms independently selected from nitrogen, oxygen, or sulfur, to form a spirocyclic group;
R³ is hydrogen, 6-10 membered aryl, or 5-10 membered heteroaryl having 1 to 4 heteroatoms independently selected from nitrogen, oxygen or sulfur, wherein R³ is optionally substituted with one or more R^{x} groups;
each R^{x} is independently selected from halogen, OH, lower alkyl, lower alkoxy, lower haloalkyl, lower haloalkoxy, or CN; and
Z is a suitable leaving group;
and
(b) converting the compound of formula G-1 to a compound of formula I.

20. The method of claim 19 wherein conversion of the compound of formula F-1 to a compound of formula G-1 comprises a Suzuki reaction.

21. The method of claim 19 wherein conversion of the compound of formula G-1 to a compound of formula I comprises contacting the compound of formula G-1 with an amine or an alkali metal azide, followed by reduction.

22. A method for preparing a compound of formula D-1: 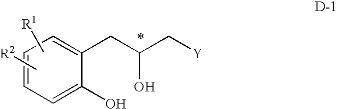
wherein:
R¹ and R² are each independently hydrogen, chlorine, fluorine, CN, —OH, C₁₋₈ alkyl, C₁-₆ perfluoroalkyl, C₁-₆ alkoxy, C₁-₆ perfluoroalkoxy, 6-10 membered aryl, 6-10 membered aryloxy, 5-10 membered heteroaryl having 1 to 4 heteroatoms independently selected from nitrogen, oxygen or sulfur, C₂-₈ alkenyl, C₁-₆ alkanesulfonamido, dialkylamino of 1 to 6 carbon atoms per alkyl moiety, C₃-₈ cycloaliphatic, or 3-8 membered heterocycloalkyl having 1 to 3 heteroatoms independently selected from nitrogen, oxygen or sulfur; or R¹ and R² when adjacent to each other may be taken together with the carbon atoms to which they are attached to form a cyclic moiety selected from a monocyclic cycloaliphatic of 3 to 8 carbon atoms, a bridged cycloaliphatic of 5 to 10 carbon atoms, a 3 to 8 membered heterocycloaliphatic having 1 to 3 heteroatoms each independently selected from nitrogen, oxygen, or sulfur, 6-10 membered aryl, or a 5-10 membered heteroaryl having 1 to 3 heteroatoms each independently selected from nitrogen, oxygen, or sulfur, wherein the monocyclic cycloaliphatic or the heterocycloaliphatic may be optionally substituted at a single carbon atom with a 3-5 membered cycloalkyl ring or a 3-5 membered heterocycloalkyl ring having 1-2 heteroatoms independently selected from nitrogen, oxygen, or sulfur, to form a spirocyclic group; and
Y is Br, Cl, or I; comprising the steps of:
(a) providing a compound of formula A: 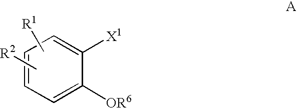
wherein:
R¹ and R² are each independently hydrogen, chlorine, fluorine, CN, —OH, C₁₋₈ alkyl, C₁-₆ perfluoroalkyl, C₁-₆ alkoxy, C₁-₆ perfluoroalkoxy, 6-10 membered aryl, 6-10 membered aryloxy, 5-10 membered heteroaryl having 1 to 4 heteroatoms independently selected from nitrogen, oxygen or sulfur, C₂-₈ alkenyl, C₁-₆ alkanesulfonamido, dialkylamino of 1 to 6 carbon atoms per alkyl moiety, C₃-₈ cycloaliphatic, or 3-8 membered heterocycloalkyl having 1 to 3 heteroatoms independently selected from nitrogen, oxygen or sulfur; or R¹ and R² when adjacent to each other may be taken together with the carbon atoms to which they are attached to form a cyclic moiety selected from a monocyclic cycloaliphatic of 3 to 8 carbon atoms, a bridged cycloaliphatic of 5 to 10 carbon atoms, a 3 to 8 membered heterocycloaliphatic having 1 to 3 heteroatoms each independently selected from nitrogen, oxygen, or sulfur, 6-10 membered aryl, or a 5-10 membered heteroaryl having 1 to 3 heteroatoms each independently selected from nitrogen, oxygen, or sulfur, wherein the monocyclic cycloaliphatic or the heterocycloaliphatic may be optionally substituted at a single carbon atom with a 3-5 membered cycloalkyl ring or a 3-5 membered heterocycloalkyl ring having 1-2 heteroatoms independently selected from nitrogen, oxygen, or sulfur, to form a spirocyclic group;
R⁶ is a suitable hydroxyl protecting group; and
X¹ is halogen,
(b) converting a compound of formula A to a compound of formula C 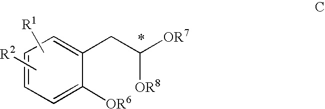
R¹ and R² are each independently hydrogen, chlorine, fluorine, CN, —OH, C₁₋₈ alkyl, C₁-₆ perfluoroalkyl, C₁-₆ alkoxy, C₁-₆ perfluoroalkoxy, 6-10 membered aryl, 6-10 membered aryloxy, 5-10 membered heteroaryl having 1 to 4 heteroatoms independently selected from nitrogen, oxygen or sulfur, C₂-₈ alkenyl, C₁-₆ alkanesulfonamido, dialkylamino of 1 to 6 carbon atoms per alkyl moiety, C₃-8 cycloaliphatic, or 3-8 membered heterocycloalkyl having 1 to 3 heteroatoms independently selected from nitrogen, oxygen or sulfur; or R¹ and R² when adjacent to each other may be taken together with the carbon atoms to which they are attached to form a cyclic moiety selected from a monocyclic cycloaliphatic of 3 to 8 carbon atoms, a bridged cycloaliphatic of 5 to 10 carbon atoms, a 3 to 8 membered heterocycloaliphatic having 1 to 3 heteroatoms each independently selected from nitrogen, oxygen, or sulfur, 6-10 membered aryl, or a 5-10 membered heteroaryl having 1 to 3 heteroatoms each independently selected from nitrogen, oxygen, or sulfur, wherein the monocyclic cycloaliphatic or the heterocycloaliphatic may be optionally substituted at a single carbon atom with a 3-5 membered cycloalkyl ring or a 3-5 membered heterocycloalkyl ring having 1-2 heteroatoms independently selected from nitrogen, oxygen, or sulfur, to form a spirocyclic group;
R⁶ is a suitable hydroxyl protecting group;
R⁷ is a hydroxyl protecting group; and
R⁸ is hydrogen or a hydroxyl protecting group,
(c) reacting the compound of formula C with a hydrogen bromide to produce a compound of formula C′: 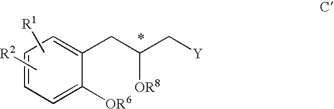
R¹ and R² are each independently hydrogen, chlorine, fluorine, CN, —OH, C₁₋₈ alkyl, C₁-₆ perfluoroalkyl, C₁-₆ alkoxy, C₁-₆ perfluoroalkoxy, 6-10 membered aryl, 6-10 membered aryloxy, 5-10 membered heteroaryl having 1 to 4 heteroatoms independently selected from nitrogen, oxygen or sulfur, C₂-₈ alkenyl, C₁-₆ alkanesulfonamido, dialkylamino of 1 to 6 carbon atoms per alkyl moiety, C₃-₈ cycloaliphatic, or 3-8 membered heterocycloalkyl having 1 to 3 heteroatoms independently selected from nitrogen, oxygen or sulfur; or R¹ and R² when adjacent to each other may be taken together with the carbon atoms to which they are attached to form a cyclic moiety selected from a monocyclic cycloaliphatic of 3 to 8 carbon atoms, a bridged cycloaliphatic of 5 to 10 carbon atoms, a 3 to 8 membered heterocycloaliphatic having 1 to 3 heteroatoms each independently selected from nitrogen, oxygen, or sulfur, 6-10 membered aryl, or a 5-10 membered heteroaryl having 1 to 3 heteroatoms each independently selected from nitrogen, oxygen, or sulfur, wherein the monocyclic cycloaliphatic or the heterocycloaliphatic may be optionally substituted at a single carbon atom with a 3-5 membered cycloalkyl ring or a 3-5 membered heterocycloalkyl ring having 1-2 heteroatoms independently selected from nitrogen, oxygen, or sulfur, to form a spirocyclic group;
Y is Br, Cl, or I;
R⁶ is a suitable hydroxyl protecting group; and
R⁸ is hydrogen or a suitable hydroxyl protecting group,
and
(d) if the R⁶ group of formula C′ is a hydroxyl protecting group, then further comprising the step of removing the protecting group to produce a compound of formula D where Y is Br.
